(19) United States
(12) Patent Application Publication
(10) Pub. No.: US 2026/0183518 A1
(43) Pub. Date: Jul. 2, 2026

(54) INTRAVASCULAR CATHETER WITH INTEGRATED GUIDE STRUCTURE

(71) Applicant: VenoCare, Inc., Doral, FL (US)

(72) Inventors: Amir BELSON, Savyon (IL); Raul LEYTE-VIDAL, Doral, FL (US); Daniel PICO, Doral, FL (US)

(21) Appl. No.: 18/854,955

(22) PCT Filed: Apr. 7, 2023
(86) PCT No.: PCT/US2023/065556

§ 371 Date: Oct. 7, 2024

Related U.S. Application Data
(60) Provisional application No. 63/328,732, filed on Apr. 7, 2022.

(51) Int. Cl. A61M 25/06; A61B 5/15

(52) U.S. Cl. CPC A61M 25/065; A61B 5/150992; A61M 25/0662; A61M 2025/0681

(57) ABSTRACT

Intravascular access is achieved by introducing a catheter having a guide element recess placed to receive the contoured distal end of a guide element. The guide element may be advanced along an outer surface of an access needle to form a loop beyond the distal end of the needle. The deployed guide element may form a partial or complete loop once beyond the distal end of the needle. After the catheter has been properly positioned, the access needle and guide element may be removed by automatic withdrawal leaving the catheter in place within a blood vessel and available for use.

### CROSS REFERENCE TO RELATED APPLICATIONS

This patent application claims priority to U.S. Provisional Patent Application No. 63/328,732, filed Apr. 7, 2022, titled “INTRAVASCULAR CATHETER WITH INTEGRATED GUIDE STRUCTURE”, which is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### STATEMENT AS TO FEDERALLY SPONSORED RESEARCH

This invention was not made with Government support.

### FIELD

The present invention relates generally to methods and systems for performing vascular access. More particularly, the present invention provides a catheter and needle assembly with an integrated guide element or structure for transcutaneous insertion of the catheter into a patient's arterial or venous vasculature.

### BACKGROUND

One common type of vascular access is called venipuncture. Venipuncture refers generally to the process of obtaining intravenous access for any one of a variety of purposes, including intravenous infusion, therapy, blood sampling, and the like. In the hospital, for example, venipuncture is commonly used to place a small intravenous catheter for delivering intravenous fluids, drug delivery, blood sampling and the like.

While venipuncture and other forms of vascular access in relatively healthy patients can be a simple matter, such access is often needed in patients who are not healthy and may have small, tortuous, collapsed, fragile, and/or difficult to locate arteries and/or veins. In such patients, venipuncture and other forms of vascular access can be very challenging, particularly to less experienced phlebotomists, paramedics, nurses, and other health care practitioners.

In addition to difficult access, many vascular catheter placement systems can result in accidental punctures and/or accidental needle contamination during or after placement of the intravascular catheter. Still further, some conventional catheter placement devices employ relatively complex deployment handle movements that lead to increases both cost and complexity. Additionally, conventional handle placement and movements can obscure the presence and status of the needle and guide structure or guide element components of the tool, thus making use of the insertion tool less intuitive.

For these reasons, it would be desirable to provide improved methods, systems, and tools for deploying intravascular catheters using needles and guide structures. It would be particularly desirable to provide simplified deployment systems and assemblies having fewer components and, even more desirably, to provide components which are clearly visible to the user and configured to be utilized and manipulated in a straightforward, intuitive manner. At least some of these objectives will be met by the various embodiments that follow.

### SUMMARY OF THE DISCLOSURE

In general, in one embodiment, an intravascular catheter assembly includes a handle having a proximal end and a distal end, a slot in a surface of the handle having a distal end and a proximal end, an actuation button on the handle coupled to a needle carrier, a tubular catheter body having a distal end, a guide element cutout in the distal end, a proximal hub, and a lumen extending between the proximal end and the distal end, the proximal hub of the catheter body coupled to the distal end of the handle, a blood control valve within the proximal hub; the blood control valve configured to be selectively advanced within the proximal hub, an access needle disposed in the tubular catheter body lumen having a tissue-penetrating distal tip extending distally beyond the distal end of the tubular catheter body and a proximal end coupled to the needle carrier, a guide element having a proximal end and a distal end, the guide element positioned within the catheter body lumen and adjacent to an outer wall of the access needle, a distal end portion of the guide element is shaped to conform with the guide element cutout of the catheter, wherein the access needle and the guide element may be withdrawn together from the tubular catheter body after the distal most portion of the guide element has been advanced from the tubular catheter body lumen along an outer wall of the access needle to a position distal to the tissue-penetrating distal tip of the access needle, and a slide coupled to the proximal end of the guide element so that distal advancement of the slide advances the distal tip end portion of the guide element from a position within the guide element cutout distally along the outer wall of the access needle.

This and other embodiments can include one or more of the following features. When the slide is in a distal most position a distal portion of the guide element can curve beyond the tissue penetrating distal tip of the access needle. When the slide is in a distal most position a distal portion of the guide element can curve from a position above the access needle, beyond the tissue penetrating distal tip of the access needle to a position below the access needle. The angle of the curve formed by the distal portion of the guide element relative to the tissue penetrating distal tip of the access needle can be less than 360 degrees. The angle of the curve formed by the distal portion of the guide element relative to the tissue penetrating distal tip of the access needle can be more than 270 degrees. The angle of the curve formed by the distal portion of the guide element relative to the tissue penetrating distal tip of the access needle can be less than 270 degrees. The angle of the curve formed by the distal portion of the guide element relative to the tissue penetrating distal tip of the access needle can be more than 180 degrees. The slide can be disposed over a proximal region of the access needle. The intravascular catheter assembly can further include a housing attached to a proximal end of the access needle, wherein the slide is disposed over the housing. The access needle can be fixedly secured to a distal end of the housing and the tubular catheter body is detachably secured to the distal end of the housing, wherein the slide advances the guide element beyond the distal end of the catheter and wherein the housing, access needle and guide element may be detached and removed from the catheter after the catheter is in place. A proximal region of the catheter can be disposed within the housing and wherein the proximal end of the catheter and the proximal end of the access needle can be configured to be engaged by the slide to advance the catheter and needle in tandem with the guide element relative to the housing after the guide element has been extended distally beyond the distal end of the catheter. The access needle can have a lumen and the guide element can be disposed outside of the access needle lumen. The housing can have an axial slot and the proximal end of the guide element can be connected to the slide allowing movement along the axial slot. The axial slot can be closed over distal and proximal regions of the access needle. A link between the guide element and the slide can be disposed in the slot with a length of travel defined by closed ends of the slot. The guide element can be slidably disposed within a guide structure within the catheter body lumen. The guide structure can maintain the guide element in a position along an upper most portion of the access needle. The guide structure can be a pair of features extending from the interior wall of the catheter body and protruding into the catheter body lumen. When the guide structure is positioned between the pair of features extending the guide structure can be positioned along a superior aspect of an exterior wall of the access needle. The guide structure can have a distal most portion adjacent to the guide element cutout and a proximal portion more than 1 cm from the guide element cutout. The guide structure can have a distal most portion adjacent to the guide element cutout and a proximal portion wherein the guide structure can be continuous along the interior wall of the catheter body lumen from the proximal end to the distal end. The tubular catheter body can have a proximal hub with a blood control valve, wherein the access needle slidably can extend through the blood control valve. The slide can have a distal face which mates with a proximal face of the needle carrier when the slide is fully advanced distally to extend the guide element. The slide detachably can lock to the proximal end of the needle carrier when the distal face mates with the proximal face. The intravascular catheter can further include a first configuration, a second configuration and a third configuration. A first configuration when the slide is in a proximal most position, the distal end of the guide element can be in the guide element cutout and the tissue penetrating distal tip of the needle can be distal to the distal most end of the tubular catheter body. A second configuration when the slide is in a distal most position, the distal most end of the guide element can be beyond the guide cutout and can be curved beyond the tissue penetrating distal tip of the needle. A third configuration, the slide can be in a position proximal to the distal most position and the needle and the guide element can be at least partially retracted into the housing. In the first configuration, the distal tip of the access needle can extend beyond the distal end of the tubular catheter body by a distance in a range from 0.1 mm to 20 mm, and a proximal end of the slide can be retracted from the proximal end of the tubular catheter body by a distance in a range from 10 mm to 100 mm. In the second configuration, the length of the guide element extending beyond the guide cut out of the tubular catheter body can be from 5 mm to 100 mm. The guide element can have a length greater than a width and a width that is greater than a thickness. The guide element can be made entirely or partially of a polymeric material, a PTFE, a Nylon. The guide element can be made entirely or partially of metal, comprising Nitinol, comprising Elgiloy or similar materials. The guide element can further include an upper surface conforming to a curvature of an interior wall of the catheter body lumen and a lower surface conforming to a curvature of an outer wall of the access needle. The catheter can be configured for use as a catheter component of an infusion set, a catheter component in a blood collection set, a catheter component in a safety winged blood collection set, or a catheter component in an intravenous catheter.

In general, in one embodiment, a method of accessing a blood vessel of a patient includes advancing a tissue penetrating distal tip of an access needle of an intravascular catheter assembly into a blood vessel of the patient, the access needle having a flat surface along an upper portion, a lower portion, a right side portion or a left side portion, advancing a guide element distal tip from a first position within a guide cutout in a distal portion of a catheter hub to a second position where a portion of the guide element elongate body is advanced along the flat surface of an access needle to a position beyond the distal most portion of the catheter hub, advancing the guide structure to a third position wherein the distal tip of the guide element is positioned beyond the tissue penetrating distal tip of the access needle and along the blood vessel of the patient, advancing a distal portion of the catheter hub in the blood vessel along the guide structure, and
automatically withdrawing the needle and the guide structure into the intravascular catheter assembly.

This and other embodiments can include one or more of the following features. The method of accessing a blood vessel can further include ceasing to perform the step of advancing a tissue penetrating distal tip of an access needle of the intravascular catheter assembly when bleed back is observed in the intravascular catheter assembly. The method of accessing a blood vessel can further include ceasing to perform the step of advancing a tissue penetrating distal tip of an access needle when a distal most portion of a catheter hub of the intravascular catheter assembly is within the blood vessel of the patient. The method of accessing a blood vessel can further include ceasing to perform the step of advancing a tissue penetrating distal tip of an access needle when a distal most portion of a guide structure of the intravascular catheter assembly is within the blood vessel of the patient. After performing the step of advancing the guide structure beyond the tissue penetrating distal tip of the access needle a distal portion of the guide element can curve beyond the tissue penetrating distal tip of the access needle. After performing the step of advancing the guide structure beyond the tissue penetrating distal tip of the access needle a distal portion of the guide element can curve from a position above the access needle, beyond the tissue penetrating distal tip of the access needle to a position below the access needle. The angle of the curve formed by the distal portion of the guide element relative to the tissue penetrating distal tip of the access needle can be less than 360 degrees. The angle of the curve formed by the distal portion of the guide element relative to the tissue penetrating distal tip of the access needle can be more than 270 degrees. The angle of the curve formed by the distal portion of the guide element relative to the tissue penetrating distal tip of the access needle can be less than 270 degrees. The angle of the curve formed by the distal portion of the guide element relative to the tissue penetrating distal tip of the access needle can be more than 180 degrees. Advancing the guide element from a first position can include advancing a proximal guide tube into a lumen of a distal guide tube in communication with a needle carrier, wherein a proximal end of the guide element can be coupled to the proximal guide tube.

In general, in one embodiment, an intravascular access device includes a handle having a proximal end and a distal end, a slot extending from the proximal end to the distal end, a catheter having a proximal catheter hub and a distal catheter lumen, the proximal catheter hub releasably engaged to the distal end of the handle, an access needle extending proximally from within a needle carrier through the catheter lumen, the access needle having at least one flat surface extending longitudinally along a perimeter of the access needle, and a tissue-penetrating tip extending distally beyond the catheter lumen, at least one guide tube within the handle, the guide tube having a lumen therethrough and a distal end in communication with a lumen extending through a distal segment of the needle carrier, a guide element having at least one longitudinal flat surface, the guide element extending from the proximal end of the handle through the at least one guide tube and needle carrier, a distal portion of the guide tube disposed within the catheter lumen, wherein the longitudinal flat surface of the guide element is in contact with the at least one flat surface of the access needle, and a slide extending through the slot, the slide in communication with a proximal end of the guide element so that distal advancement of the slide advances a distal tip portion of the guide element from a position adjacent to a distal end of the catheter lumen along the at least one flat surface of the access needle.

This and other embodiments can include one or more of the following features. The at least one flat surface of the access needle can extend longitudinally down an exterior surface of the access needle. The guide element can be disposed through a path defined by the at least one flat surface of the access needle, a distal guide tube coupled to the needle, and a proximal guide tube axially aligned with the at least one flat surface of the access needle. The at least one guide tube can be aligned parallel to a plane of the at least one flat surface of the access needle. When the slide is in a distal most position, a distal portion of the guide element can curve beyond the tissue-penetrating distal tip of the access needle. When the slide is in a distal most position, a distal portion of the guide element can curve from a position defined by a plane of the at least one flat surface of the access needle, beyond the tissue-penetrating distal tip of the access needle. The intravascular access device can further include an access needle lumen, wherein a plane of the at least one flat surface of the access needle can be perpendicular to a radius of the access needle lumen. The intravascular access device can further include a blood control valve disposed within the proximal catheter hub, the blood control valve having a body with a distal end and a proximal end and a lumen within the body can extend from the proximal end to the distal end. The distal end of the blood control valve can include a surface extending across the lumen, the surface can include a plurality of slots. The proximal end of the blood control valve body can include a perimeter surface on a proximal end configured to engaged with a portion of a Luer to advance the blood control valve distally within the catheter hub displace the plurality of slots. The proximal end perimeter can be continuous on a single plane. The proximal end perimeter can include a recess into an exterior of the valve body. The recess can be configured to accommodate a distal end of a distal guide tube, a segment of the guide element body, or distal end of the needle carrier. In use, when the needle carrier is in a distal most position, the needle carrier can be proximal to the blood control valve proximal end without contacting the blood control valve. The tissue-penetrating tip can define a distal terminus of a beveled surface of the access needle. The at least one flat surface can be offset on the perimeter of the access needle at a degree relative to the position of the tissue-penetrating tip. The intravascular access device can further include an actuation button coupled to the needle carrier, and an actuation element exerting a force on the needle carrier towards the proximal end of the handle, wherein when the actuation button is depressed the actuation element can displace the needle carrier and access needle toward the proximal end of the handle. A plane of the at least one flat surface of the access needle can be perpendicular to any radial extending outward from a center of the access needle. The catheter can be configured for use as a catheter component of an infusion set, a catheter component in a blood collection set, a catheter component in a safety winged blood collection set, or a catheter component in an intravenous catheter. The intravenous access device can further include a spool of guide element in communication with the proximal end of the intravascular device, wherein a length of the guide element can be contained within the spool. The guide element can be made entirely or partially of a metallic material, polymeric material, or a combination thereof.

In general, in one embodiment, an intravascular catheter assembly includes a handle having a proximal end and a distal end, a slot in a surface of the handle having a distal end and a proximal end, a spring loaded actuation button on the handle coupled to a needle carrier, a tubular catheter body having a distal end, a guide element cutout in the distal end, a proximal end, and a lumen extending between the proximal end and the distal end, the proximal end of the catheter body coupled to the distal end of the handle, an access needle disposed in the tubular catheter body lumen having a multi-facet tissue-penetrating distal tip extending distally beyond the distal end of the tubular catheter body and a proximal end coupled to a needle carrier within the handle, a guide element comprising an elongate body extending distally from a guide element tip conformed to operably couple with the guide element cutout, the guide element positioned within the catheter body lumen and adjacent to an exterior surface of the access needle, wherein the access needle may be withdrawn from the tubular catheter body after the guide element tip has been advanced from the tubular catheter body lumen along an outer wall of the access needle to a position distal to the tissue-penetrating distal tip of the access needle, and a slide coupled to the proximal end of the guide element so that distal advancement of the slide advances the guide element tip distally from a position within the guide element cutout.

This and other embodiments can include one or more of the following features. When the slide is in a distal most position a distal portion of the guide element can curve beyond the tissue penetrating distal tip of the access needle. When the slide is in a distal most position a distal portion of the guide element can curve from a position above the access needle, beyond the tissue penetrating distal tip of the access needle to a position below the access needle. A proximal side of the guide element tip can be configured to contact the guide element cutout. The access needle can include a flat surface positioned axially along a length of the access needle exterior surface. The guide element elongate body can extend proximally within the catheter body lumen adjacent to an axial flat surface along the access needle exterior. The guide element tip can include a geometry configured to complement the guide element cutout. The guide element tip can be affixed to a distal end of the guide element elongate body. The intravascular catheter assembly can further include a housing attached to a proximal end of the access needle, wherein the slide can be disposed over the housing. The access needle can be fixedly secured to a distal end of the housing and the tubular catheter body can be detachably secured to the distal end of the housing. The slide can advance the guide element beyond the distal end of the catheter and wherein the housing, access needle and guide element may be detached and removed from the catheter after the catheter is in place. The intravascular catheter assembly can further include a blood control valve positioned within a hub at a proximal end of the tubular catheter body, the blood control valve can include a valve body and a lumen, wherein a surface covers a distal end of the lumen the surface having a plurality of slits. The intravascular catheter assembly can further include a proximal guide tube axially aligned with a distal guide tube, wherein the guide element elongate body can extend through the proximal guide tube, distal guide tube, and catheter body lumen. The proximal end of the guide element can be coupled to an interior of a proximal guide tube. The proximal guide tube can be axially aligned with a distal guide tube coupled to the needle carrier. The guide element can be slidably disposed along the flat surface of the access needle within the catheter body lumen. The tubular catheter body can have a proximal hub with a blood control valve, wherein the access needle slidably can extend through the blood control valve. The slide can have a distal face which mates with a proximal face of the needle carrier when the slide is fully advanced distally to extend the guide element. The intravascular catheter assembly can further include a first configuration, a second configuration and a third configuration. A first configuration when the slide is in a proximal most position wherein the guide element distal tip can be in the guide element cutout and the tissue penetrating distal tip of the needle is distal to the distal most end of the tubular catheter body. A second configuration when the slide is in a distal most position wherein the guide element distal tip can be beyond the guide cutout and curved beyond the tissue penetrating distal tip of the needle. A third configuration when the slide is in a position proximal to the distal most position and the needle can be at least partially retracted into the housing. The catheter can be configured for use as a catheter component of an infusion set, a catheter component in a blood collection set, a catheter component in a safety winged blood collection set, or a catheter component in an intravenous catheter.

In general, in one embodiment, an intravascular catheter assembly includes a catheter lumen extending distally from a proximal hub, the catheter lumen comprising a guide element cut out in a distal end of the catheter lumen, an access needle disposed in the catheter lumen and having a flat surface positioned axially along a length of the access needle, a guide element disposed between the flat surface of the needle and the interior of the catheter lumen, a slide coupled to a proximal segment of the guide element so that distal advancement of the slide advances a molded distal tip of the guide element from a first position at the guide element cut out to a second position wherein a distal segment of the guide element curves beyond the tissue penetrating distal tip of the access needle, wherein the slide has a distal face which mates with a proximal face of the needle carrier when the slide is fully advanced distally to extend the guide element.

This and other embodiments can include one or more of the following features. The slide detachably can lock to the proximal hub when the distal face of the slide mates with the proximal face of the needle carrier. The guide can have an elongate body extending distally from the molded tip, the elongate body comprising a flat surface in communication with the flat surface of the access needle. The guide element can be a polymer.

In general, in one embodiment, a method of accessing a blood vessel of a patient includes advancing a tissue penetrating tip of an access needle of an intravascular catheter assembly into a blood vessel of the patient, advancing a formed distal tip of a guide element from a first position within a guide element cutout in a distal portion of a catheter lumen, where a distal portion of the guide structure forms a uniform transition to an exterior of the catheter lumen, to a second position where a portion of the guide structure is advanced along a flat surface of the access needle beyond the tissue penetrating tip, the flat surface positioned axially along outer wall of the access needle, advancing a distal portion of the catheter hub in the blood vessel along the guide element, and withdrawing the needle into the intravascular catheter assembly.

This and other embodiments can include one or more of the following features. The method of accessing a blood vessel can further include ceasing to perform the step of advancing a tissue penetrating distal tip of an access needle of the intravascular catheter assembly when bleed back is observed in the intravascular catheter assembly. The method of accessing a blood vessel can further include advancing a blood control valve positioned within the catheter hub. The formed distal tip of the guide element and a distal most portion of the guide element can form an atraumatic tip after being advanced beyond the tissue penetrating tip. Advancing the formed distal tip of the guide element can include advancing a slider distally within a slot of a handle, wherein the slider can be coupled to a proximal guide tube, wherein the guide element can extend through a lumen of the proximal guide tube.

In general, in one embodiment, an intravascular catheter includes a hub having a proximal end and a distal end, a tubular body extending from the hub distal end to a distal tip, the tubular body having an outer wall and an inner wall, a lumen within the hub and the tubular body bounded by the tubular body inner wall, the lumen having a guide element cutout in a distal end, a guide element having a formed distal tip configured to couple with the guide element cutout, and an access needle having a longitudinal flat surface along a length of an exterior of the needle, wherein the guide element comprises a flat surface adjacent to the flat surface of the needle.

This and other embodiments can include one or more of the following features. The intravascular catheter can further include a needle carrier within a handle, the needle carrier coupled to a proximal end of the access needle. The intravascular catheter can further include a proximal guide tube slidably coupled with a distal guide tube, wherein a guide element elongate body can extend proximally from the formed distal tip through a lumen formed by the distal guide tube and proximal guide tube. The intravascular catheter can further include a slide operably coupled to the proximal guide tube, wherein the slide can be configured to advance the formed distal tip by advancing the proximal guide tube.

In general, in one embodiment, an intravascular access device includes a handle having a proximal end and a distal end, a slot extending from the proximal end to the distal end, a catheter having a catheter lumen extending distally from a catheter hub, the catheter hub releasably engaged to the distal end of the handle, an access needle extending distally through the catheter lumen from a needle carrier, the access needle having at least one flat surface extending axially along the access needle body, and a tissue-penetrating tip, a guide element configured to extend through a lumen comprising the catheter lumen and a guide tube positioned within the handle proximal to the needle carrier, the guide element comprising a formed distal tip configured to engage a guide element cutout at a distal end of the catheter lumen, and a slide extending through the slot, the slide in communication with a proximal segment of the guide element, wherein the slide is configured to advance the guide element distally along the at least one flat surface of the access needle.

This and other embodiments can include one or more of the following features. The guide element can include an elongate body extending proximally from the formed distal tip. The guide element can include an elongate body extending proximally from the formed distal tip through a lumen comprising the catheter lumen and a guide tube proximal to the needle carrier. The guide element can include a distal segment having a flat surface adjacent to the at least one flat surface of the access needle. The guide element distal end can be configured to curve when advanced beyond the tissue-penetrating tip. The at least one flat surface of the access needle can include a lateral edge proximal to the tissue penetrating tip. The intravascular access device can further include a blood control valve positioned within the catheter hub. The blood control valve can include a distal segment with one or more channels configured to increase a volume of the catheter hub. A proximal segment of the blood control valve can be configured to accommodate a distal end of the needle carrier. The at least one flat surface of the access needle can extend along an exterior of the access needle at a degree relative to a position of the tissue-penetrating tip. The intravascular access device can further include an actuation button coupled to the needle carrier.

In general, in one embodiment, an intravascular catheter assembly includes a hub having a proximal end and a distal end, a tubular body extending from the hub distal end to a distal tip, and a blood control valve positioned within the hub, the blood control valve comprising a distal surface configured to accommodate a flow of fluid therethrough when the blood control valve is advanced distally within the hub.

This and other embodiments can include one or more of the following features. The blood control valve can include a recess in a proximal perimeter configured to accommodate a needle carrier. A guide element cutout at the distal tip of the tubular body can be configured to couple with a guide element distal tip. The catheter hub can include an actuation element configured to open the distal surface of the blood control valve.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a perspective view of an embodiment of an intravascular access device having a catheter, guide element and needle assembly.

FIG. 1B is a perspective view of an embodiment of an intravascular access device having a catheter, guide element and needle assembly.

FIG. 2A is a cross section view of the intravascular access device of FIG. 1A showing the catheter, guide element and needle assembly, actuation button, support rail and slider in the proximal position.

FIG. 2B is a cross section view of the intravascular access device of FIG. 1B showing the catheter, guide element and needle assembly, actuation button, support rail, first guide tube and second guide tube.

FIG. 2C is a cross section view of the intravascular access device with the catheter hub removed to show details of the blood control valve, distal guide tube and needle carrier near the distal end of the handle.

FIG. 3 is a top perspective view of the distal housing and catheter hub of FIG. 1A showing details of the needle, guide element and needle carrier within the interior of catheter hub.

FIG. 4 is an enlarged top perspective view of the distal housing and catheter hub of FIG. 3 showing details of the needle, guide element, needle carrier and needle carrier slot within the interior of the catheter hub.

FIG. 5A is a perspective view of a longitudinal cross section of the intravascular access device of FIG. 3 showing additional details of the needle carrier in relation to the actuation button and the guide element to the support rail and the access needle outer wall.

FIG. 5B is a perspective view of a longitudinal cross section of the intravascular access device of FIG. 1B showing additional details of the needle carrier in relation to the actuation button and the guide element through the needle carrier to the needle flat surface.

FIG. 6 is an upper view of a portion of the intravascular catheter of FIG. 1A proximal to the view of FIG. 4 showing additional details of the distal end of the needle carrier in relation to the guide element and support rail that are within the needle carrier longitudinal slot.

FIG. 7 is an upper view of a portion of an interior view similar to FIG. 6 that shows the proximal end of the needle carrier in relation to the slider, guide element and support rail.

FIG. 8 is an upper perspective view of a distal portion of the intravascular access device of FIGS. 1A and 3 showing the catheter hub interior including the needle carrier, the needle, the guide element and the support rail.

FIG. 9 is perspective proximal view of the components seen in FIG. 8.

FIG. 10A is an enlarged view of the distal end of the intravascular device of FIG. 1A showing the distal end of the access needle in relation to the distal tip of the catheter with a guide element cut out and the distal end of the guide element within the guide element cut out.

FIG. 10B is an enlarged view of the distal end of the intravascular device of FIG. 1B showing the distal end of the access needle in relation to the distal tip of the catheter with a guide element engagement feature and the transition from a needle flat surface to a needle bevel surface.

FIG. 11 is a distal end on view of a cross section of the vascular access device of FIG. 10A showing the relationship of the catheter inner wall, the guide element and the access needle outer wall.

FIG. 12 is a distal end on view of a cross section of the vascular access device of FIG. 10B showing the alignment of the guide element route through the guide tubes and on top of the needle flat surface.

FIG. 13 is a perspective view of the needle carrier, first guide tube, second guide tube, and orientation of the guide element route.

FIG. 14 is an enlarged view of the central portion of the view of FIG. 11 showing a recessed portion of the catheter lumen wall, the upper surface of the guide element adjacent to the lumen wall recessed portion and the guide element lower surface adjacent and conforming to the access needle outer wall.

FIG. 15 is an enlarged view of the central portion of the view of FIG. 11 similar to FIG. 14 providing additional explanation of the access needle outer wall, the catheter lumen wall in relation to the various contours of the guide element positioned between these structures of the intravascular device.

FIGS. 16A-16D are enlarged views of the central portion of an intravascular device of FIG. 1B providing additional explanation of the orientation and arrangement of the guide element relative to the needle.

FIGS. 17A and 17B are enlarged views of the distal tip of the catheter, distal tip of the needle, and distal tip of the guide element oriented at a lateral position relative to the needle.

FIG. 17C is a perspective view of a configuration for alignment of the needle flat surface, needle carrier, and guide tubes that form the guide element route at a clocked position other than on top of the needle surface.

FIG. 17D is an enlarged view of FIG. 17C and illustrates the guide wire transition through the distal end of the needle carrier and into the second guide tube.

FIG. 18 is a perspective view of the distal end of the catheter and access needle of FIG. 10A and FIG. 1A after the slider has been moved from a proximal position as shown in FIG. 1A to a distal position where the guide element is advanced distally along an outer wall of the access needle to form a loop in a position distal to the tip of the access needle.

FIGS. 19A to 19C are an enlarged views of the guide element and distal end of the catheter lumen.

FIG. 20 is an enlarged perspective view of the guide element flat surface extending through the distal end of the catheter tip and beyond the tissue-penetrating tip of the needle

FIG. 21 is a perspective view of the guide element oriented lateral to the needle in an extended configuration.

FIG. 22 is an enlarged view of the guide element loop.

FIGS. 23A to 23C are enlarged views of homeostasis valves according to embodiments of the present invention.

FIGS. 24A and 24B are enlarged views of the catheter hub and distal end of the intravascular access device handle, as described herein.

FIGS. 25A and 25B are enlarged views of the catheter hub, distal guide tube and distal end of the intravascular access device handle, as described herein.

FIGS. 26A and 26B are enlarged views of a coupling with a peripheral medical device to the catheter hub, as described herein.

FIG. 27 is an enlarged view of a catheter assembly, as described herein.

### DETAILED DESCRIPTION

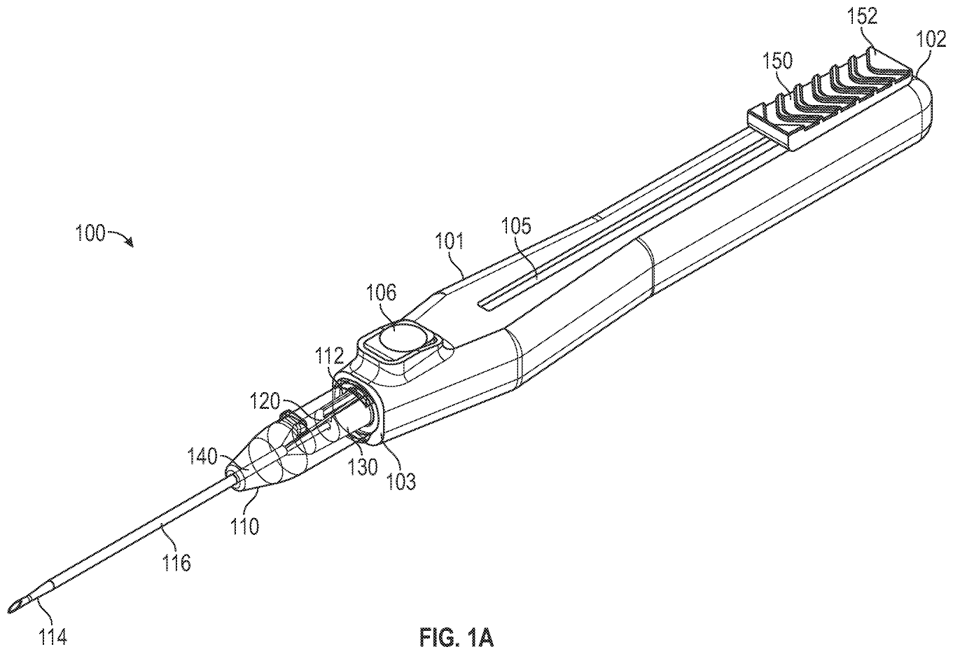

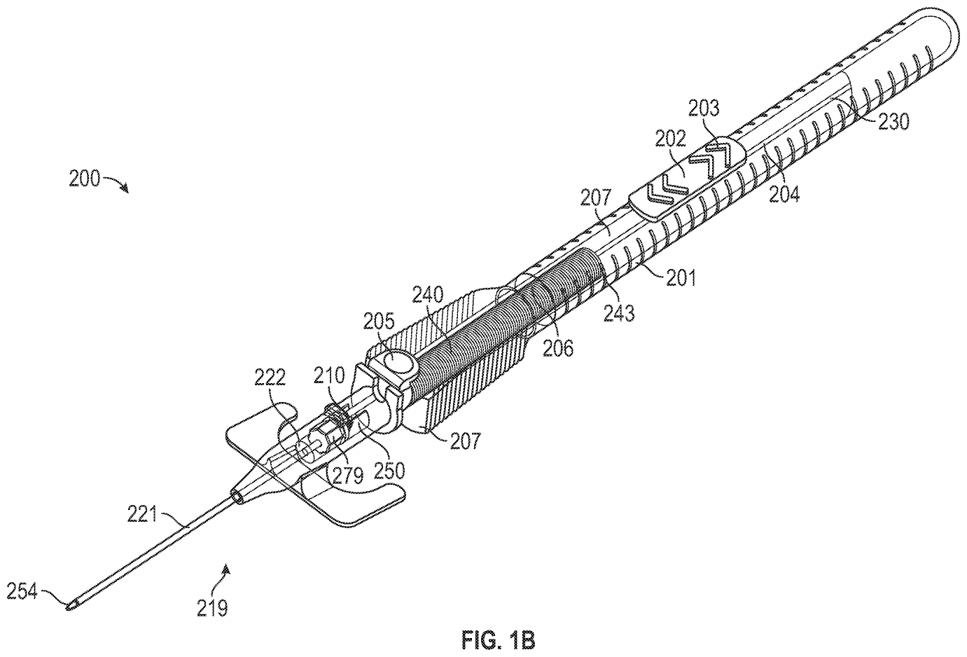

FIG. 1A is a perspective view of an embodiment of an intravascular access device 100 having a catheter hub 110, guide element 120 and needle assembly. In this configuration, the intravascular catheter with integrated guide structure or guide element is ready for use to position the catheter within a vessel. The catheter hub 110 can be coupled to the distal end 103 of the handle 101 and an access needle 140 extending from a needle carrier 130 through a catheter lumen 116 extending distally from the catheter hub 110. The catheter hub 110 is transparent in this view in order to see the position of the needle carrier 130 and guide element 120.

The handle or housing 101 has a proximal end 102 and a distal end 103. A slot 105 runs along the length providing access for the slide 150 to engage with the guide element 120 and advance the guide element tip distally from within a guide element cutout in the distal end of the catheter lumen 116. The slide 150 includes a grip 152 and is shown in the proximal most position which is used when the needle 140 is positioned in the catheter 110 and ready for insertion into the vasculature of a patient. In some examples, the catheter hub 110 has a distal end 114 and a proximal end 112 and an interior lumen 116 along the length. The catheter proximal end 112 is adapted and configured to engage with the handle distal end 103. The catheter proximal end 112 is also configured to engage with peripheral elements such as conventional medical tubing connections once inserted and the handle 101 is removed. For example, the catheter proximal end 112 may comprise features to engage with a luer lock, fluid coupling or other suitable medical connector or fitting.

FIG. 1B is a perspective view of an example of an intravascular access device 200 with certain elements shown as translucent to allow visibility of internal components. In this view, the intravascular access device has a catheter assembly 219 in communication with a distal end 207 of a handle 201, a guide element 210 traversing through a needle carrier 240. An actuation element 206 is visible through this example of a translucent handle 201. The actuation element can be configured to selectively displace the needle carrier 240 when the actuation button 205 is engaged. For example, the actuation element 206 can be a spring coiled around the needler carrier 240 and positioned between an interior wall of the distal portion of the handle to a needle carrier collar 243 as shown here. A slide 202 extends through a slot 207 in the top portion of the handle 201. The slide 202 includes a grip 203 accessible on an exterior surface of the handle 201 and an arm 204 extending through the slot 207 of the handle to a proximal end of a first guide tube 230.

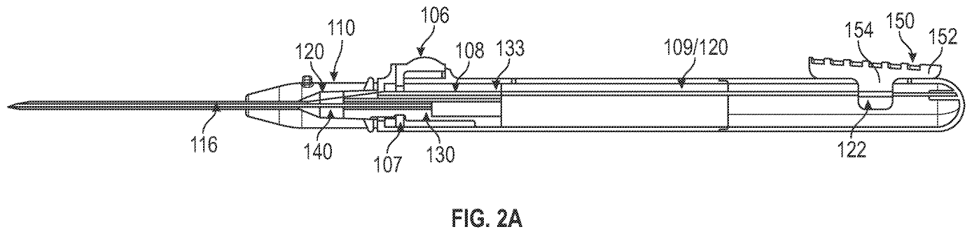

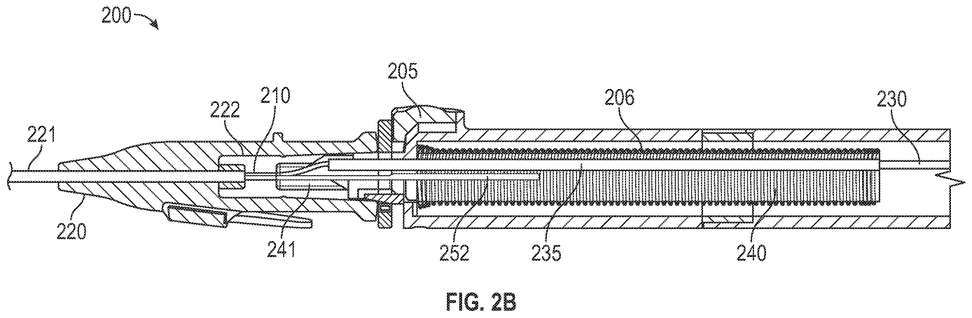

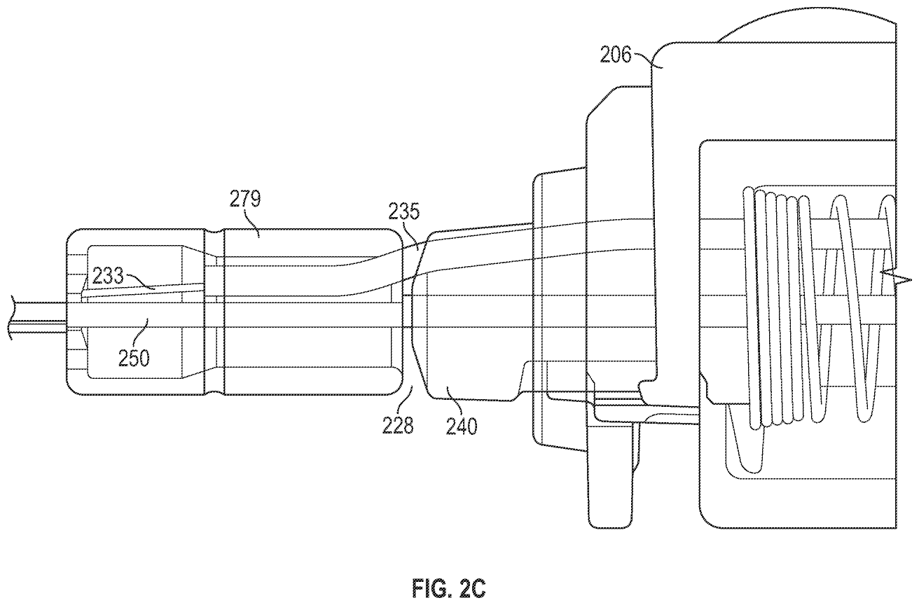

The intravascular access device illustrated in FIG. 1B is shown in a ready to use configuration. The slide 202 is positioned near the proximal end of the handle 201 and the arm 204 is in communication with the guide element 210 near the proximal end of the handle. The slide 202 can advance the guide element 210 distally as the slide may be in operable communication with the proximal end or segment of the guide element 210. The guide element 210 can have an elongate body 233 extending proximally into the handle from a distal tip with additional details described, for example, in FIG. 2B.

In some examples, the catheter assembly 219 may be deployed after the access needle 250 and distal end of the guide element 210 facilitate the catheter placement in the vessel. When the catheter 220 is deployed, an actuation button 205 shown near the distal end of the handle 201 can be depressed release the actuation element 206 to displace the needle carrier 240 and the needle 250 into the handle 201. In some examples, the actuation button is configured to release the actuation element and withdraw (e.g., proximally displace) the needle carrier 240, access needle 250 and guide element proximally. In any of the examples described herein, the withdraw of the access needle 250 and the guide element 210 (e.g., distal end of the guide element) occur simultaneously. For example, when the actuation button 205 is depressed and the actuation element 206 displaces the needle carrier 240 proximally, the access needle is withdrawn into the handle 201 and the guide element distal end may also be withdrawn proximally into the handle 201. In some examples, the needle 250 is withdrawn entirely into the handle 201. In some examples, the guide element 210 is withdrawn entirely into the handle 201.

Also shown in FIG. 1B, a blood control valve 279 can be positioned within the catheter hub 222 and can be configured to control a flow of fluid (e.g., blood) through the catheter assembly. The blood control valve 279 can prevent a flow of blood from the vessel through the catheter assembly 219 until the valve is opened at a distal end. For example, the catheter assembly may be deployed or uncoupled from the handle after access into the vessel is established and the blood control valve 279 can prevent blood from flowing out of the catheter assembly until a peripheral element (e.g., male portion of a luer lock) has been attached to the catheter hub and the blood control valve 279 is advanced distally within the catheter hub 222.

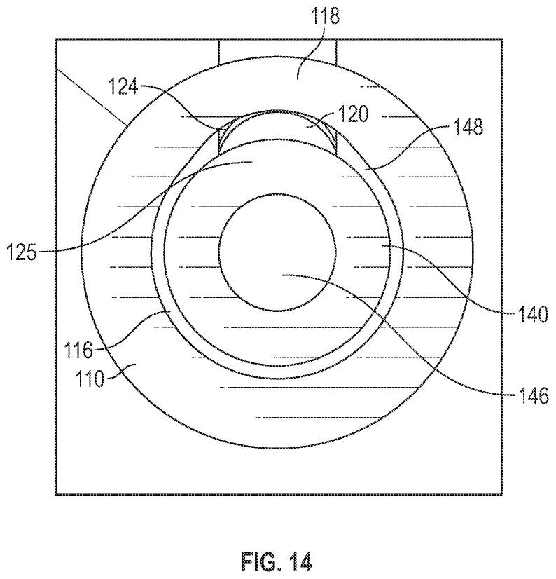

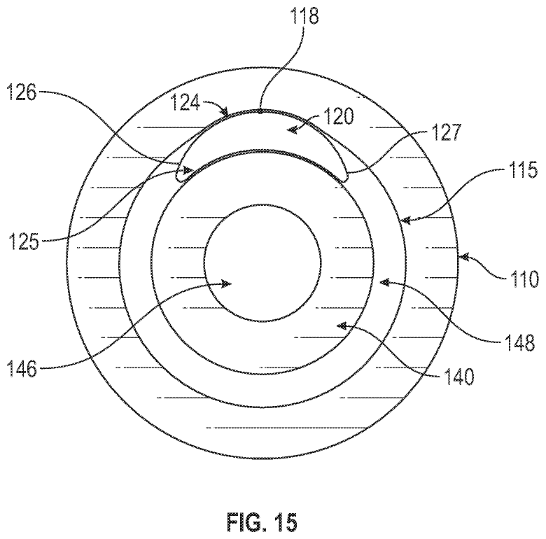

FIGS. 2A to 2C are cross section views of examples of an intravascular access device. FIG. 2A is a cross section view of the intravascular access device showing the catheter hub 110, guide element 120 and needle carrier 130, actuation button 106, support rail 109 and slide 150 in the proximal position. This is a “ready to use” configuration. The guide element proximal end 122 is attached to the slide tab 154. The guide element 120 extends along the support rail 109 beyond the needle carrier distal end 132, into and along the catheter lumen 116. The guide 120 is between the needle outer wall 148 and the catheter lumen inner wall 115. As a result, when the slide 150 is advanced along the slot 105, the guide element is advanced along the support rail 109, along and beyond the distal end of the catheter and the needle forming an atraumatic loop or partial loop or curve to cover the needle sharp distal end 144. (see FIGS. 14 and 15).

The catheter lumen 116 is also visible in this view. The needle 140 has a sharp distal end 144 used to penetrate into the skin and vessel wall. The access needle proximal end 142 terminates within the needle carrier 130. When the activation button is released, the activation element 108 moves the needle carrier 130 towards the housing proximal end 102. The movement of the needle carrier 130 within the handle 101 is long enough so that the sharp distal end 144 of the needle 140 is completely within the housing 101. The needle carrier 130 also includes a support rail slot 138 along its length sized to receive the support rail 109.

An actuation element 108 is included but not shown in this view in order to see the details of the needle carrier 130. The actuation element 108 may be a spring such as a coil spring or a wave spring or other suitable compression element. When the actuation button 106 is depressed, the actuation button latch 107 disengages from the needle carrier distal groove 134, when then allows the actuation element 108 to expand and propel the needle carrier 130 along with needle 140 towards the handle proximal end 102.

FIG. 2B is a cross section view of an intravascular access device 200 as shown in FIG. 1B. The guide element 210 comprises a distal end, not shown, and an elongate body extending proximally through the catheter and catheter hum 222 before continuing to extend through a distal guide tube 235 and then through the concentric lumen of a proximal guide tube 230. The proximal guide tube 230 and the distal guide tube 235 are generally positioned within the handle and provide additional support for the guide element extending therethrough. For example, the guide element elongate body can extend through the lumen formed by the proximal guide tube 235 and the distal guide tube 230 such that when the slider advances the guide element distally, the guide tubes can be configured to prevent buckling or otherwise support to increase a column strength of the guide element elongate body extending therethrough. In some examples, the guide element 210 is further routed through the catheter proximal hub 222 and catheter lumen 221 after passing through the needle carrier lumen 221 within the distal portion of the needle carrier 240. The needle carrier lumen 221 has a slope to facilitate a transition of the guide element 210 from the distal end of the second guide tube 235 through the distal end of the needle carrier 240 to contact an exterior surface of the needle 250. The guide element 210 continues through the catheter between the exterior surface of the needle 250 and an interior surface of the catheter lumen 221.

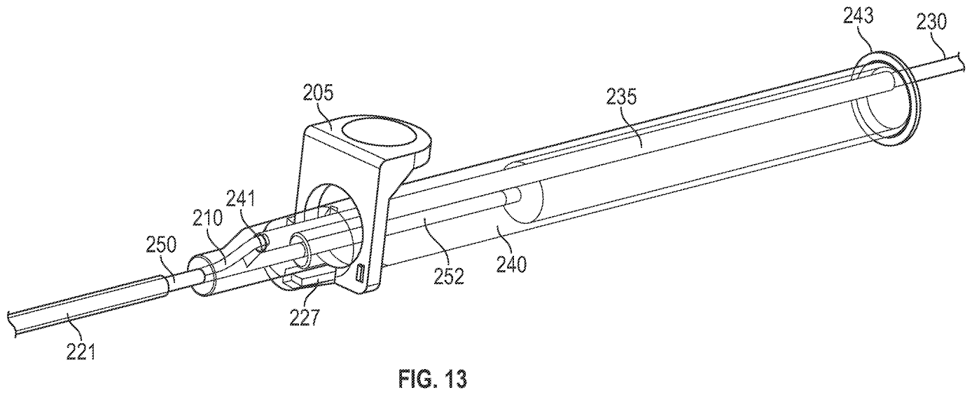

The distal end of the proximal guide tube 230 can be partially and slidably inserted into a distal guide tube 235 providing concentric lumens defining a route of the guide element within the handle. In an embodiment, the distal end of the first guide tube and the proximal end of the second guide tube may interlock with one another (See FIG. 13). For example, a channel around an interior circumference of the second guide tube may correspond with a toroidal ridge around the exterior surface of the first guide tube such that when the ridge sufficiently enters the channel, the first guide tube and second guide tube may be restrained in a position relative to one another. The interlocking configuration of the first guide tube and the second guide tube may provide retention of the needle carrier 240 after the actuator button 205 is depressed allowing the actuator element 206 to force the needle carrier 240 towards the proximal end of the handle 201. For example, as the needle carrier is biased towards the proximal end of the handle, the second guide tube will slide over the first guide tube towards the proximal end of the handle until the distal end of the first guide tube interlocks with the distal end of the second guide tube.

Alternatively, the proximal end of the second guide tube may continue to slide over the first guide tube to engage an interlocking element positioned near the proximal end of the first guide tube.

In some examples, the guide element elongate body can be coupled to the proximal guide tube 235. For example, the guide element elongate body may be affixed, crimped, or otherwise connected to a proximal end of the proximal guide tube 235 such that the slider can engage the proximal guide tube 235 to advance the guide tube-catheter combination providing a corresponding distal advancement of the guide element distal tip from the guide element cutout of the catheter.

FIG. 2C is another example of an intravascular device and shows details of a distal end of the handle and elements positioned distal to the handle or extending therethrough. The catheter hub has been removed from this view to highlight the blood control valve 279 and routing of the guide element elongate body 233 through the distal guide tube 235 as it transitions from the handle to the flat surface of the access needle 250. The distal guide tube 235 is shown having a tapered distal segment. For example, a distal segment of the distal guide tube 235 slopes towards the needle to route the guide element elongate body 233 through an interior of the blood control valve 279. Also seen in FIG. 2C is a space or gap between the proximal end of the blood control valve 279 and the distal end of the needle carrier 240. In some examples, the needle carrier 240 may have a length or configuration to support the space between the distal end of the needle carrier 240 and the blood control valve 279. Accordingly, the needle carrier may not contact the blood control valve 279 in use.

In some examples, a distal guide tube (e.g., FIG. 2C element 235) may comprise a lumen extending through the guide tube from a proximal end to a distal end. The guide tube may have a tubular lumen with a linear axis. In some examples, the guide tube lumen may have a lumen with a substantially linear axis. In some examples, the guide tube may have a lumen with a segment defined by a linear axis of the lumen where the lumen continues distally to another segment with a different axis (e.g., sloped). The configuration of the guide tube may be based on the routing of the guide element elongate body from inside of the handle through a transition to the flat surface of the access needle within the catheter lumen. In some examples, the guide tube (e.g., distal guide tube) may be in communication with or adjacent to the needle carrier.

In any example, the distal guide tube may be adjacent to the needle carrier. For example, a distal guide tube may be couple to the needle carrier, integrated in the needle carrier, affixed to the needle carrier, attached to the needle carrier, seated within or along the needle carrier, or a combination thereof.

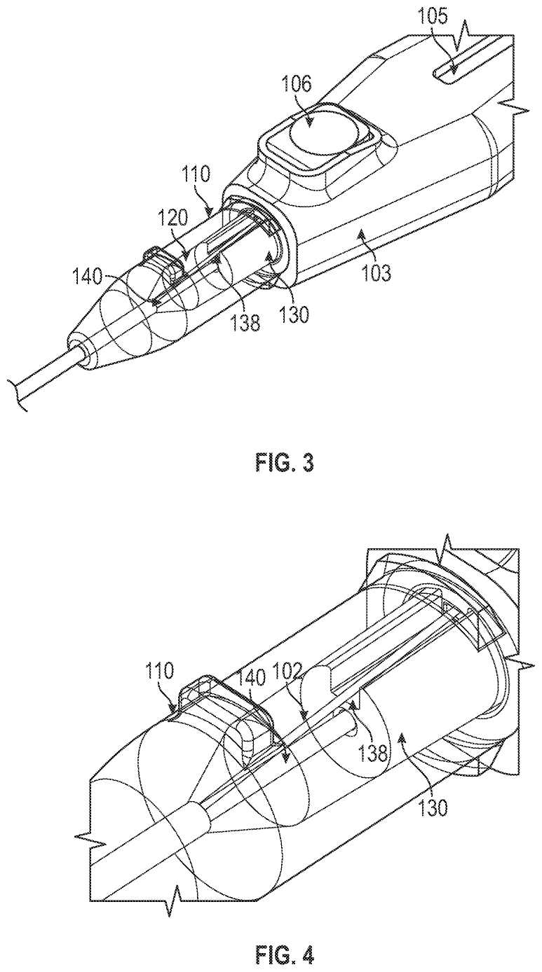

FIG. 3 is a top perspective view of the distal housing 103 and catheter hub 110 of FIG. 1A showing details of the needle 140, guide element 120 and needle carrier 130 within the interior of catheter hub 110. The guide element 120 is shown exiting the distal end of a support rail and entering the space between the needle outer wall and the inner wall of the catheter lumen. In some examples, the guide element elongate body may be routed through one or more guide tubes, as described herein. In some examples, the guide tube elongate body may be routed through a channel in the needle carrier.

FIG. 4 is an enlarged top perspective view of an exemplary distal housing 103 and catheter hub 110 of FIG. 3 showing details of the needle 140, guide element 120, needle carrier 130 and needle carrier support rail slot 138 within the interior of the catheter hub 110. In this view the needle 140 is shown exiting the catheter lumen 116 and entering the needle carrier lumen 136. The proximal end of needle 140 terminates within and is secured to the needle carrier 130.

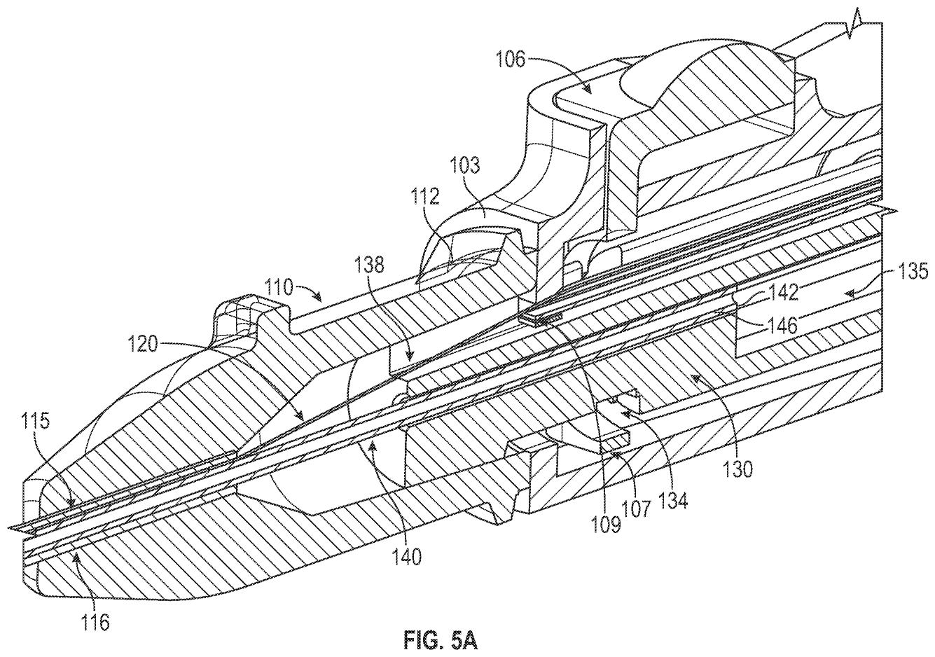

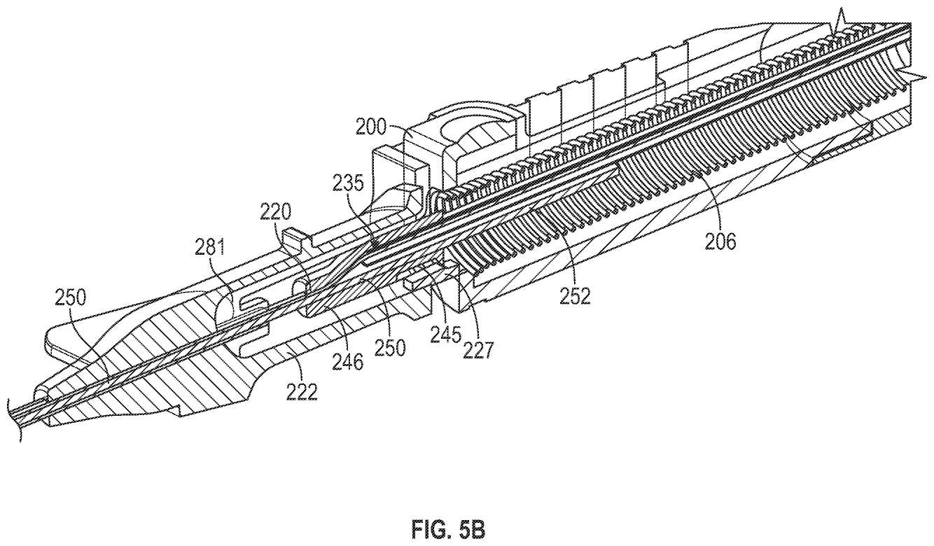

FIG. 5A is a perspective view of a longitudinal cross section of the intravascular access device 100 of FIG. 3 showing additional details of the needle carrier 130 in relation to the actuation button 106 and the guide element 120 to the support rail 109 and the access needle outer wall 148. The catheter hub proximal end 112 is shown coupled to the housing or handle distal end 103. Additional details of the guide element relative to other components may also be appreciated. The guide element 120 extends along the support rail 109, the distal end of the slot 138 into the gap between the needle outer wall and the catheter lumen wall 115. Additionally, the needle 140 is shown in relation to the catheter lumen proximal end 116 and the needle carrier lumen 146. The needle proximal end 142 terminates in the needle carrier 130 into a blood return chamber 135. All or a portion of the needle carrier 130 or the handle 101 may be transparent in order to allow for visual detection of fluid or blood return or bleed back once the needle distal end 144 has entered a target vessel.

FIG. 5A also shows the relationship of the activation latch 107 to the associated needle carrier groove 134. When in the loaded or cocked configuration, the latch 107 would engage with the groove 134 to provide hold back force against the activation element 108. Depression of the activation button 106 will disengage the latch 107 from the needle carrier slot 134 allowing the spring force or stored energy of the activation element 108 to propel the needle carrier 130 towards the handle proximal end 102. This view also shows the termination of the support rail 109 within the housing distal end 103.

FIG. 5B is a perspective view of a longitudinal cross section of the intravascular access device 200 of FIG. 1B illustrating the route of the guide element 210 sloped from the second guide tube 235 through the distal portion of the needle carrier 240 to an exterior surface of the needle 250. Details of the proximal segment of the needle 250 retained within the needle carrier 240 are visible and generally parallel to the first and second guide tubes.

Here, the relationship of the actuator button 205 to the associated needle carrier groove 245. When in the loaded or cocked configuration, the latch 227 would engage with the groove 245 to provide hold back force against the activation element 205. Depression of the activation button 205 will disengage the latch 227 from the needle carrier slot 207 allowing the spring force or stored energy of the activation element 206 to propel the needle carrier 240 towards the handle proximal end.

FIG. 5B also illustrates the relationship of components defining the route of the guide element through an interior of the intravascular access device 200. In particular, the route of the guide element is defined by the concentric and colinear lumens of the first guide tube 230 and second guide tube 235. The first and second guide tubes are parallel and coplanar with the surface of the needle contacted by the guide element 210. An exterior surface of the distal portion of the needle carrier is shown having a slope corresponding to the slope of the lumen therethrough. The cross section view of FIG. 5B highlights an embodiment where each of the components defining the route of the guide element align with one another whereby the guide element 210 has a one deviation, in a ready-to-use configuration, from an otherwise linear route through the interior of the intravascular access device 200. A hemostasis valve 281 (e.g., blood control valve) may be positioned adjacent to the needle carrier distal end 246. As seen in FIG. 5B, the blood control valve 281 may within the catheter proximal hub 222 and can be configured to minimize, prevent or reduce a backflow of fluid (e.g., blood) from the proximal catheter hub 222. In some examples, the guide element 220 and needle 250 may pass through the distal end of the blood control valve 281.

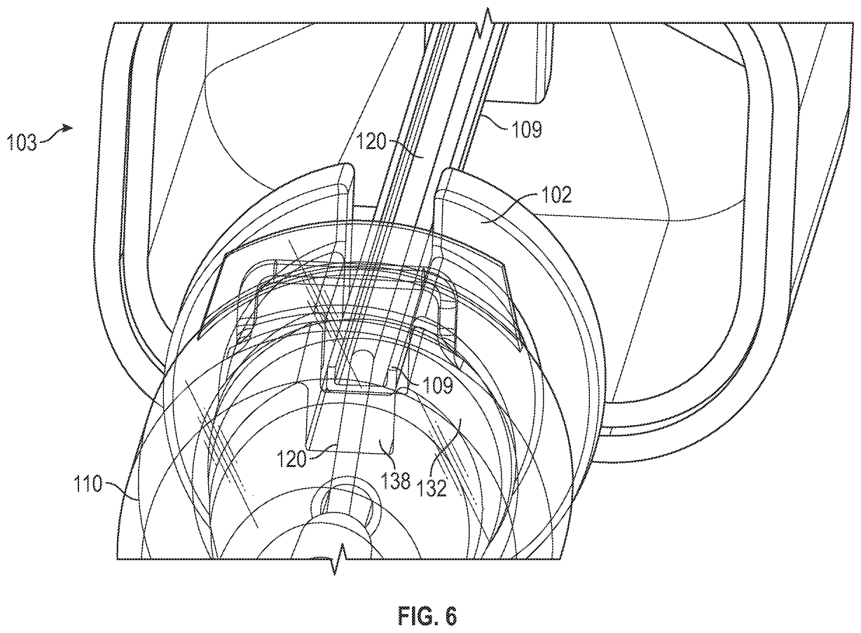

FIG. 6 is an upper view of a portion of the intravascular catheter of FIG. 1A proximal to the view of FIG. 4 showing additional details of the distal end 132 of the needle carrier 130 in relation to the guide element 120 and support rail 109 that are within the needle carrier longitudinal slot 138. The guide element 120 can be seen along the upper surface of the support rail 109, transitioning from the support rail distal end, beyond the needle carrier distal end 132 into the catheter lumen 116 along needle outer wall 148. The arrangement of the support rail 109 to the needle carrier support rail slot 138 allows the support rail to act like a monorail for the needle carrier during retraction (i.e., when the stored energy of the actuation element 108 is released. The support rail 109 also supports the guide element 120 during advancement of the slide 150.

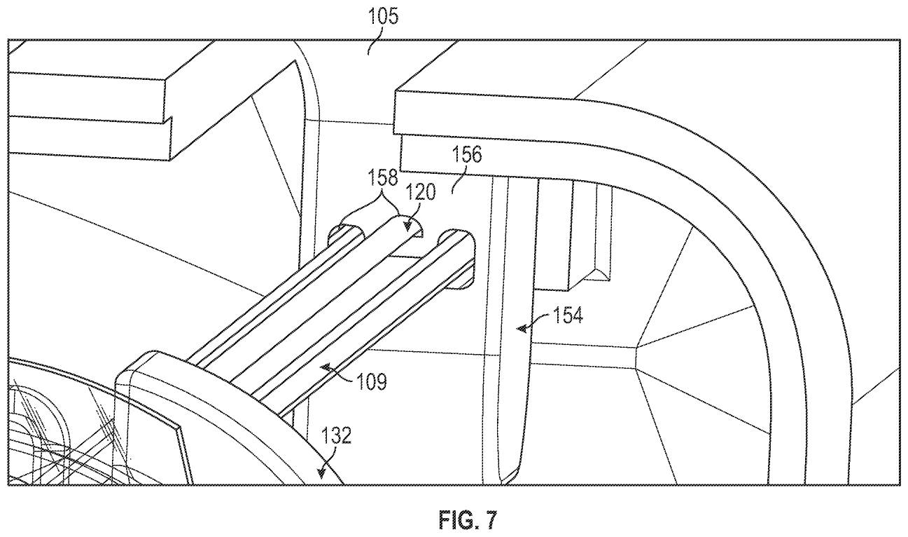

FIG. 7 is an upper view of a portion of an interior view similar to FIG. 6 that shows the proximal end of the needle carrier 132 in relation to the slide 150, guide element 120 and support rail 109. The slider tab 154 of slide 150 extends through slot 105. The slider tab includes a key plate 156 having apertures 158. The apertures 158 are sized, shaped and positioned to accommodate the guide element 120 and features of the support rail 109. Advantageously, the arrangement of the slide 150 and support rail 109 permits the support rail 109 to allow the slide 150 to translate back/forward freely. The guide element 120 has a proximal end 122 that is connected to the slide using the slider tab 154 or other suitable structure. The support rail 109 extends through the slide tab and is connected to the housing proximal end 102. Additionally, this view also shows the relationship of the needle carrier proximal end 132 which is sized and positioned so as to engage with the slide tab 154 during retraction. The rearward movement of the needle carrier will cause the needle carrier proximal end 132 to engage with and move the slide tab 154. The guide element 120 will also move proximally within the housing since the guide element 120 is terminated in the slide 150. The result of this movement (i.e., the proximal movement of the needle carrier 130) simultaneously withdraws the needle and the guide element into the interior of the handle or housing.

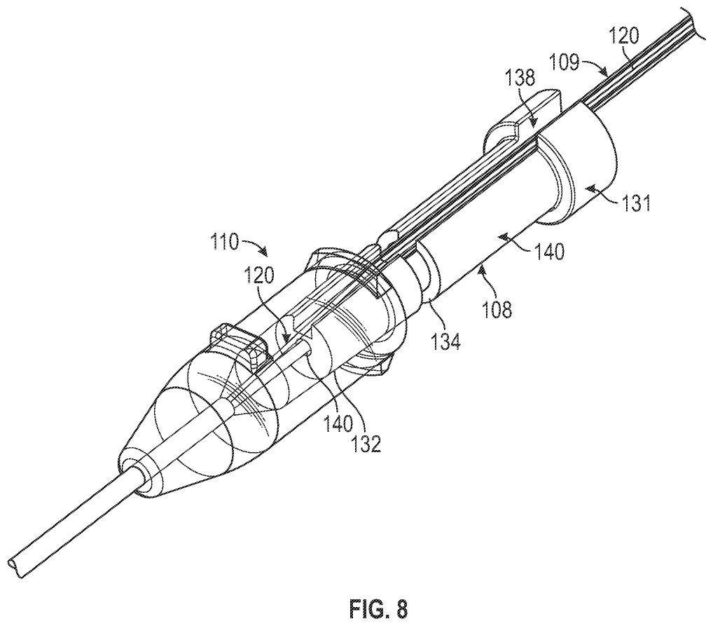

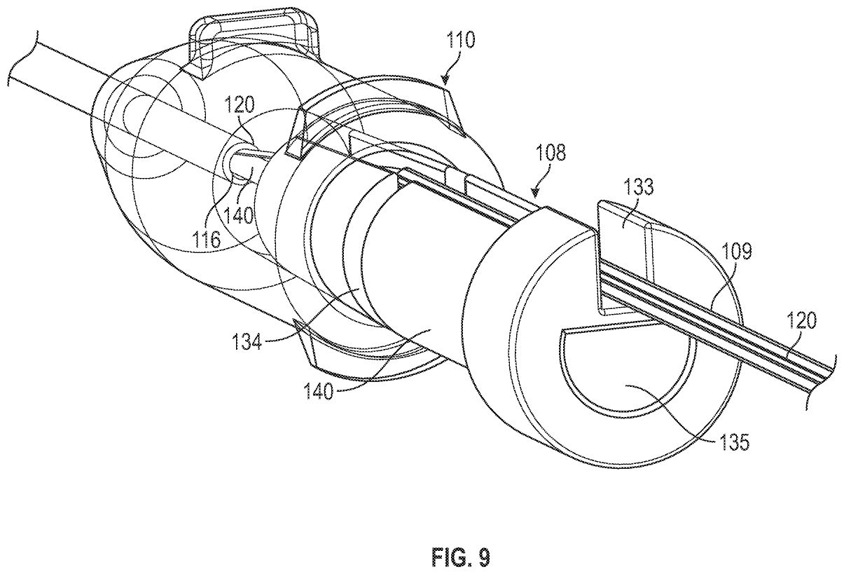

FIG. 8 is an upper perspective view of a distal portion 103 of the intravascular access device 100 of (e.g., shown in FIGS. 1A and 3) showing the catheter hub 110 interior including the needle carrier 130, the needle 140, the guide element 120 and the support rail 109. This is another view of the placement of the guide element 120 along the support rail 109, beyond the needle carrier and into the catheter lumen. This view also shows the support rail slot 138 along the length of the needle carrier 130 in relation to the needle carrier proximal end 131 and distal groove 134. FIG. 9 is perspective proximal view of the needle carrier 130 and other components seen in FIG. 8. The blood chamber 135 is visible within the needle carrier proximal end 131. A normally present cover for the chamber 135 is removed to show the interior.

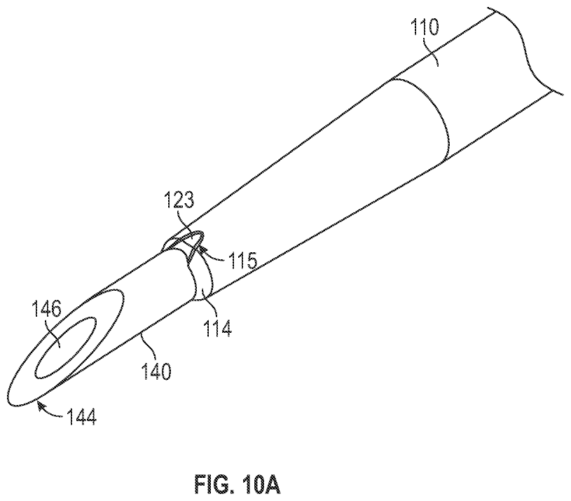

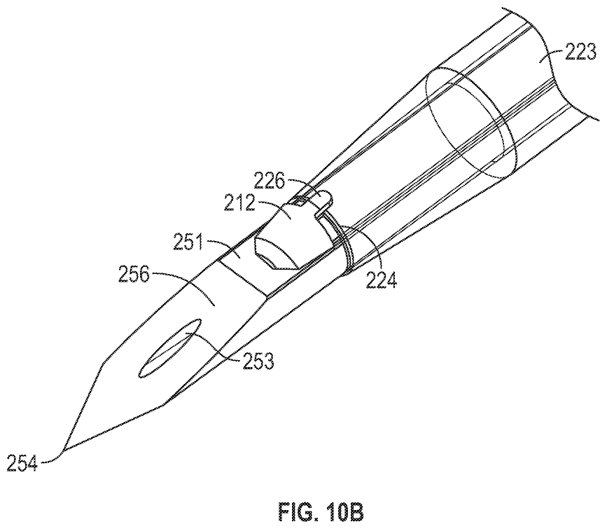

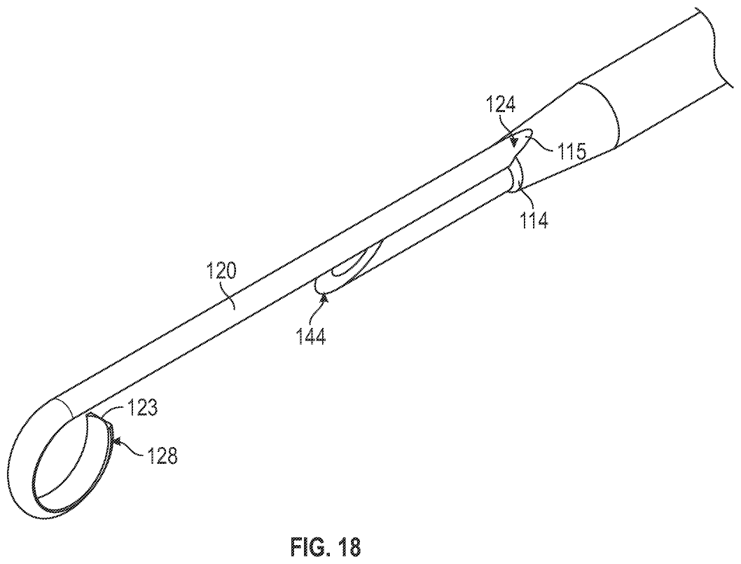

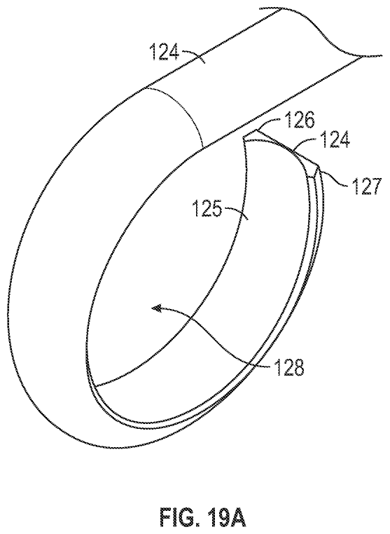

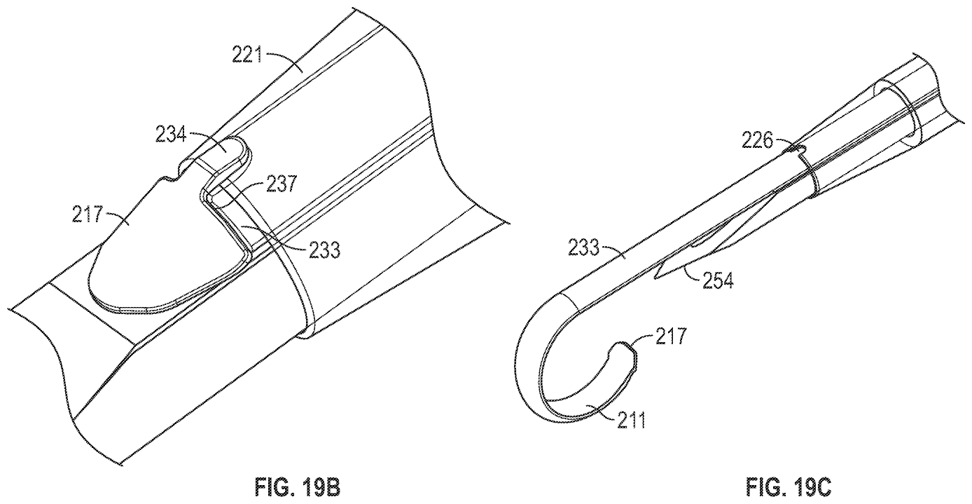

FIG. 10A is an enlarged view of the distal end of an intravascular device (e.g., 100 of FIG. 1A) showing the distal end 144 of the access needle 140 in relation to the distal tip 114 of the catheter with a guide element cut out 115 and the guide element distal end 123 of guide element 120 within the guide element cut out 115. The guide element distal end 123 can be molded/manufactured to mimic the geometry of catheter tip 114. The guide element (e.g., guide element elongate body) can extend over the needle within the catheter lumen along a flat surface extending along the needle body. For example, the guide element 120 can be positioned between catheter lumen inner wall or the inner diameter and needle outer wall or the needle outer diameter. In some examples, the guide element positioned within the needle lumen does not require additional space or enlarged catheter gauge as the needle flat surface provides a route or path for the guide element elongate body. In some examples, the guide element 120 can be composed of a polymeric material. The guide element 120 may also be pre-shaped or shape set or have a form of shape memory to form a complete loop, a partial loop or multiple loops once extended beyond the needle distal end 144. (See FIGS. 18 and 19).

In some examples, the catheter distal end comprises a guide element cut out 115 configured to couple with the guide element distal tip 123. The guide element cutout 115 can have a geometry corresponding to the guide element distal tip 123 to facilitate a smooth or continuous exterior surface from an exterior of the needle to an exterior of the catheter. The view in FIG. 10A is an example of how the shaped distal end of the guide element conforms to the catheter tip guide element cut out. This is also a view of a uniform transition between the catheter and the contour of the guide element distal end such that the catheter tip to needle transition and the circumference of the catheter tip remain smooth.

In FIG. 10B an example of a guide element distal end 212 (e.g., formed distal tip) is shown as molded/manufactured to mimic the geometry of the guide element cutout at the catheter tip 224 at the distal end of the catheter. The distal end of the catheter is shown with a guide element engagement feature 226 (e.g., guide element cutout) having a shape configured to receive a portion of the guide element distal end 212. The geometry of the catheter tip 224 is shown angled or having a slope towards the exterior surface of the needle 250. The guide element distal end 212, when in communication with the guide element cutout 226, has a slope corresponding to the slope of the catheter tip 224 resulting in a smooth transition from the exterior surface of the needle 250 to the exterior surface of the catheter 220. Also illustrated in FIG. 10B is the needle flat surface 251 and transition to the needle beveled surface 256 terminating at the tissue-penetrating tip 254. In an embodiment and in the ready-to-use configuration, the tissue-penetrating tip 254, guide element distal end 212 and catheter tip 224 may be referred to as the distal end assembly.

In use, the geometry of the distal end assembly provides a smooth transition through one or more layers of tissue lead by the tissue-penetrating tip 254. As the distal end assembly is advanced through one or more layers of tissue and the tissue-penetrating tip 254 may enter the vasculature of a patient without unnecessary damage or restriction within the tissue or vasculature.

The guide element distal end 212 (e.g., guide element formed tip) may be molded to the distal end of the guide element elongate body 223. In some examples, the guide element distal tip 212 may be coupled to the guide element elongate body 223. For example the guide element distal tip 212 can be affixed, or otherwise attached to the distal end of the guide element elongate body 223. The geometry of the guide element distal tip 212 can be configured to complement or otherwise engage the guide element cutout 226. The distal end of the catheter may have a non-uniform distal perimeter with a distal surface configured to couple (e.g., seat, receive, engage, etc.) with the guide element distal tip 212. Accordingly, the guide element distal tip may be formed or otherwise comprise an element configured to engage the cutout. For example, the guide element distal tip 212 may comprise a proximal surface configured to contact or otherwise be positioned adjacent to the distal surface of the catheter at the guide element cutout.

In some examples, the guide element can comprise a flexible distal segment configured to facilitate a change from a linear state to a curled state when the guide element is advanced distally and retracted. For example, the guide element distal segment may be configured to curl beyond the tissue penetrating tip after the formed guide element tip has been advanced from the guide element cutout. When the guide element and needle are to be retracted, the flexible distal segment of the guide element can be configured straighten or sufficiently straighten as the guide element distal tip is retracted through the catheter distal end and into the catheter lumen. In some examples, the guide element distal segment or distal end are sufficiently flexible to provide for retraction of the guide element without displacement or impact to the position of the catheter (e.g., within the vessel).

In an embodiment, the needle flat surface 251 transitions to the needle beveled surface 256. The guide element distal end can be molded to match the slope of the needle beveled surface 256 and provide a smooth continuation of the needle beveled surface slope to the exterior surface of the catheter 220.

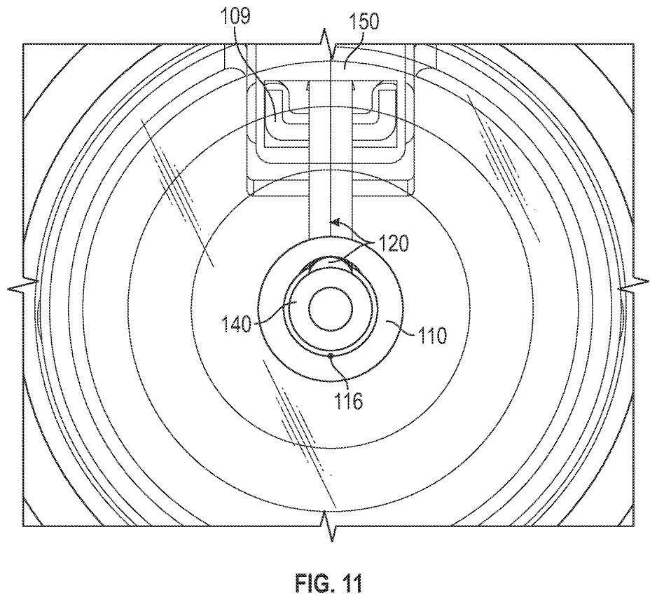

FIG. 11 is a distal end on view of a cross section of the vascular access device of FIG. 10A showing the relationship of the catheter inner wall 115, the guide element 120 and the access needle outer wall 148. The guide element is position within and translates along the space within the catheter lumen along the outer wall of the access needle and the inner wall of the catheter lumen. Optional catheter design includes groove 118 recessed into the catheter lumen inner wall that corresponds to a portion of the guide element upper surface shape 124 or contour. The catheter groove 118 assists with maintaining guide element 120 in position along the catheter and translation along the needle outer wall 148.

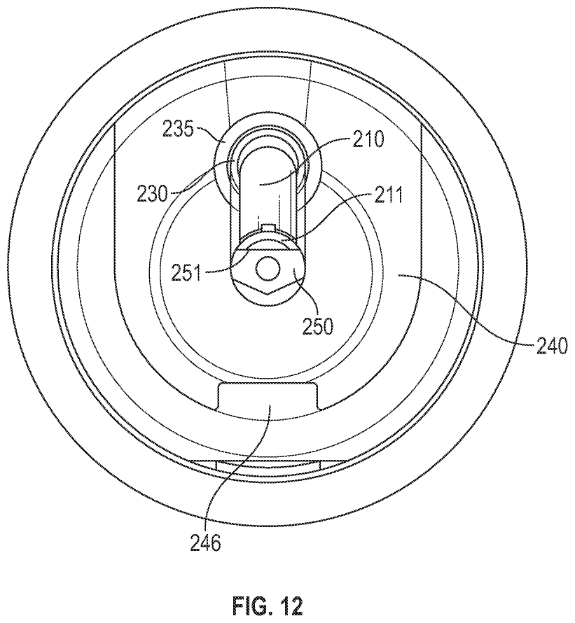

Additionally or optionally, the guiding element design of upper 124 and lower 125 surface contours may correspond to one or both of the catheter lumen interior wall and needle outer wall or to other alignment elements within the catheter lumen. Optionally, in other additional embodiments, the needle outer wall 148 may include a groove or recessed portion for embodiment where a guide element 120 is shaped and configured to operate in an inverted configuration from that shown in this view. See the enlarged views in FIGS. 12 and 13 for additional details of the catheter-guide element-needle alternative embodiments.

FIG. 12 is an enlarged view of an embodiment of the intravascular access device 200 illustrating components defining the route of the guide element. As shown in FIG. 5B, the guide element 210 is disposed through a lumen initially defined by the proximal guide tube 230 and the distal guide tube 235. The guide element 210 (e.g., guide element elongate body) continues through the distal portion of the needle carrier 240 to contact a needle flat surface 251. The alignment of the proximal guide tube 230, the distal guide tube 235 and the lumen through the distal portion of the needle carrier 240 are based on the surface of the needle contacted by the guide element 210. In this embodiment, the needle has a semicircular cross-sectional geometry whereby a segment of the needle circumference is substantially flat. The flat segment of the exterior surface of the needle is the surface in communication with guide element. FIG. 12 shows the guide element 210 also having a flat surface that is in contact with the needle flat surface 251.

In an embodiment, the needle flat surface 251 and the guide element flat surface 211 are in contact with each other. Accordingly, an exterior surface of the guide element 210 may have an arc corresponding to the curvature of the exterior surface of the needle 250. The guide element flat surface 211 may have a width corresponding to the chord line defining the needle flat surface 251 such that when they are in contact, their exterior surfaces form a substantially continuous circumference.

FIG. 13 is a perspective view of select components associated with the route of the guide element 210. In particular, the needle carrier 240 is illustrated and transparent making the second guide tube 235 within the needle carrier 240 visible. The proximal guide tube distal end 230 is obscured within the distal guide tube proximal end 235. A needle proximal end 252 retained within the needle carrier 240 can be seen below the distal guide tube 235. The guide element 210 is shown in from the distal guide tube distal end 235 and transitioning through the sloped needle carrier lumen 241. The guide element 210 is shown adjacent to the needle flat surface 251 as the needle 250 and guide element 210 traverse the catheter 220. The route of the guide element 210 above and on top of the needle flat surface 251 is aligned with the orientation of the needle flat surface 251 such that the slope of the needle carrier lumen 241 is positive and upward towards the colinear second and first guide tubes. The needle 250 is seen generally parallel to the first and second guide tubes on the same plane of the needle carrier lumen 241 and guide element 210. FIG. 13 shows examples of guide tubes having a concentric lumens generally extending along a single axis. The guide element elongate body then transitions out of the distal guide tube 235 and transitions through to the needle flat surface. In some examples, the transition of the guide element elongate body from the distal guide tube may be through the distal guide tube, through the distal portion of the needle carrier, or sloping through a space within the catheter hub, or a combination thereof.

FIG. 14 is an enlarged view of the central portion of the cross-section view of FIG. 11. This view shows an exemplary location and shape of a recessed portion 118 of the catheter lumen wall 115. This view also shows the guide element 120 in cross section with the upper surface 124 of the guide element adjacent to the lumen wall 115 recessed portion 118 and the guide element lower surface 125 adjacent and conforming to the access needle outer wall 148. The access needle 140 and needle lumen 146 are also shown within the catheter lumen 116.

FIG. 15 is an enlarged view of an alternative configuration the central portion of the cross section view of FIG. 11 but similar to FIG. 14. Those of ordinary skill will appreciate that the guide element may have any of a variety of an upper surface contour 124, a lower surface contour 125, a right side contour 127 and a left side contour 126. Still further, variations in the cross section profile or contours of the guide element 120 may allow for the guide element to extend along and occupy different portions of the space between the catheter inner wall and the needle outer wall. Additionally or optionally, one or more guide element contours may be complementary to an inner wall recess, a guide element track or one or more guide element track features on, in or within a portion of the catheter lumen inner wall or needle outer wall. In one aspect, a guide element upper surface 124 may be contoured or shaped to at least partially conform to catheter lumen inner wall. Additionally or optionally, a guide element lower surface 126 may be shaped to at least partially conform to an access needle exterior wall 148. In the same way, one or more guide element embodiments may employ variations to the right and left contours 126, 127 so as to be adapted to engage with or be complementary to other features provided along the needle or catheter lumen to allow for smooth translation of the guide element along and beyond the access needle distal end 142. In some embodiments, the distance between the guide element left side contour 126 and the guide element right side contour 127 ranges between 0.10 mm to 0.36 mm. This distance is considered the guide element width. Additionally, in still other embodiments, the distance between the guide element upper surface contour 124 and the guide element lower surface contour 125 ranges between 0.127 mm to 0.203 mm. This distance is considered the guide element thickness. In some embodiments, the guide element has a length between 100 mm and 200 mm.

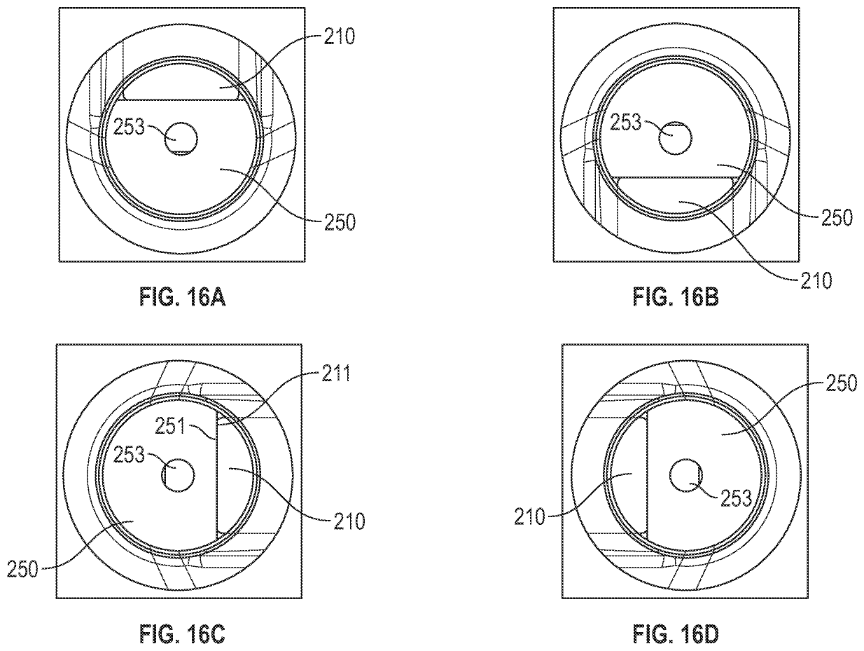

FIGS. 16A-16D are enlarged views of alternative configurations the central portion of the cross section view of the needle 250 of the intravascular access device 200 shown in FIG. 12. Detailed here are examples of different embodiments regarding the position of the guide element 210 about an outer surface of the needle 250. The guide element 210 is superior to the needle in FIG. 16A and as shown in FIGS. 1B and 12. FIG. 16B shows the guide element 210 positioned inferior relative to the needle 250. FIGS. 16C and 16D show the guide element 210 contacting the needle flat surface 251 on either lateral side of the needle 250, respectively. The needle flat surface 251 contacts the corresponding guide element flat surface 211 and when each of these flat surfaces are in contact with one another, a circumference is generally formed including the remaining exterior surface of the needle and the remaining exterior surface of the guide element. The location of the needle flat surface 251 on the perimeter of the needle defines the location of the guide element 210 relative to the needle 250 and therefore the guide element route through the first guide tube 230, the second guide tube 235, the needle carrier 240, and the catheter lumen 221.

In an embodiment, the needle 250 has a cross sectional geometry that is substantially semicircular with greater that 180 degrees of arc terminating at the flat surface. The needle flat surface 251 may be perpendicular to any conceivable radial extending outward from a center of the needle lumen 253. The tissue-penetrating tip 254 provides a static reference point in describing the location of the needle flat surface 251. For example, a location of the tissue-penetrating tip may be set at the 180° radial about the center axis of the needle. A radial position of the needle flat surface 251 can be described relative to the static location of the tissue-penetrating tip 254. For example, FIG. 16A, where the guide element 210 is superior to the needle flat surface 251, the needle flat surface 251 may be perpendicular to a 360° radial. In this example, a line drawn from the tissue penetrating tip, across the needle to a midpoint of the needle flat surface could be the diameter of the needle.

In some examples, the location of the needle flat surface 251 is described relative to a clock position. When the needle flat surface 251 is clocked and existing longitudinally on the perimeter of the needle 250, the clocked position may be described relative to the terminal end of the needle. For example, the tissue-penetrating tip 245, may be a static reference for 6 o'clock and when describing the location of the needle flat surface 254 on the perimeter of the needle, the clocked position is described considering the tissue-penetrating tip 254 as a static reference relative to the clocked location of the needle flat surface. For example, FIG. 16A illustrates an example of the needle flat surface 251 in the 12 o'clock position, FIG. 16B illustrates an example of the needle flat surface 251 at a 6 o'clock position, FIG. 16C illustrates an example of the needle flat surface 251 at a 3 o'clock position and FIG. 16D illustrates an example of the needle flat surface 251 at a 9 o'clock position. While the figures shown provide examples of the position of the needle flat surface 251, the needle flat surface 251 may be at any position at or between the examples provided.

In some examples, the needle flat surface 251 and guide element flat surface are adjacent to one another. As seen in FIGS. 16A-16D, the adjacent flat surfaces provide for a circular or substantially circular cross sections. For example, the guide element 210 has an exterior surface opposite the guide element flat surface 211 having an arc corresponding to the arc of the exterior surface of the needle. Accordingly, a space or gap between the exterior surface of the needle-guide element assembly and the interior of the catheter lumen is reduced or substantially eliminated allowing for an increased opportunity to use standard sized catheter lumens. Reducing or eliminating the gap between the needle-guide element assembly and the interior of the catheter lumen and improve insertion into a blood vessel by reducing the dilation or physical insult to the tissue caused by a larger gauge catheter. In some examples, reducing the gap or spacing between the needle-guide element assembly can reduce the amount of blood flowing along an exterior of the needle when the catheter is inserted into the vessel by improving a flow of blood through the needle lumen 253.

In some examples, the position of the flat surface of the needle can establish the route or path of the guide element elongate body. For example, if the flat surface is generally at a 12 o'clock position, the guide element elongate body may extend proximally along the top of the needle and then up to the a distal guide tube and through the interior of the handle. Where the flat surface is at a 6 o'clock position, the guide element elongate body may extend proximally along a side of the needle to a guide tube positioned along a path corresponding to the side of the needle flat surface to route the guide element elongate body within the handle.

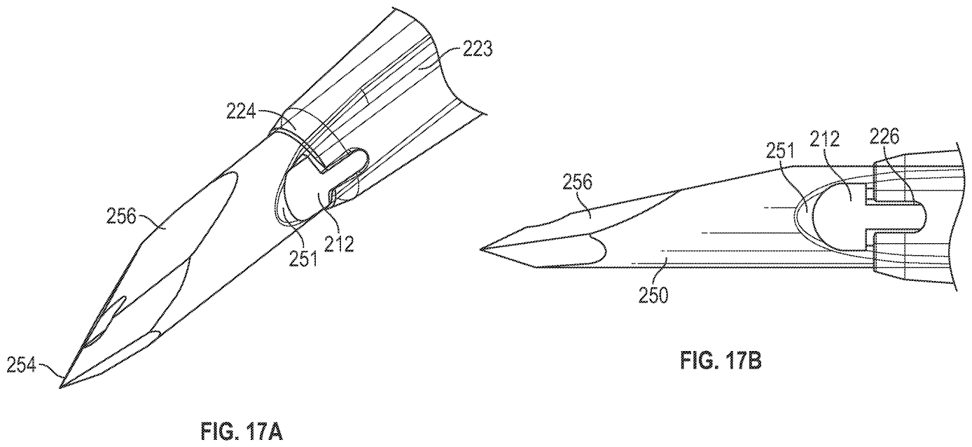

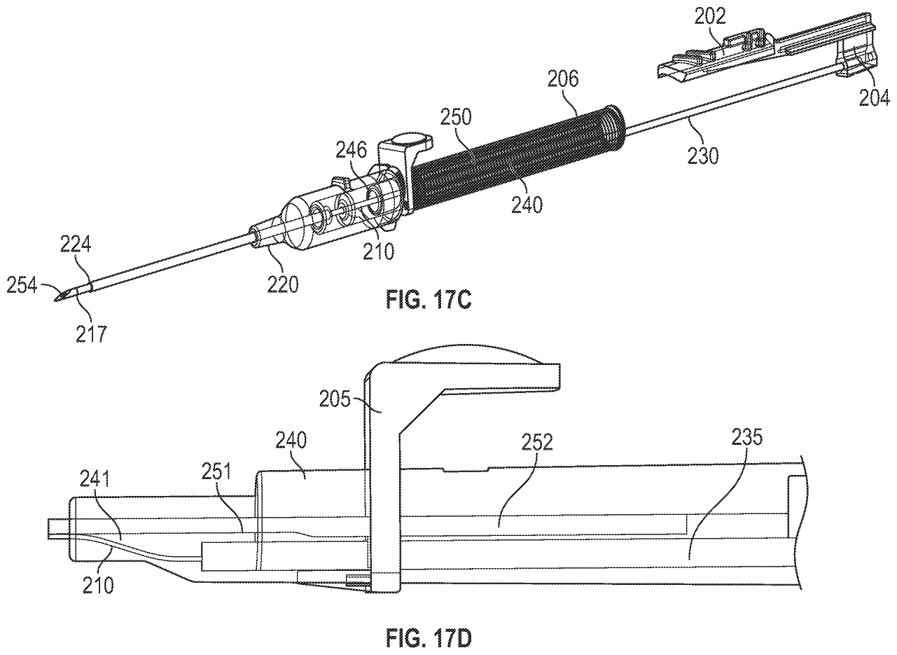

FIGS. 17A and 17B are closeup images of the distal portion of the needle 250 The needle has a tissue-penetrating tip 254 at the distal point of a needle beveled surface 256. The distal end of the catheter lumen 221 is shown having a beveled surface extending between the tissue-penetrating tip 254. In an embodiment, more than one beveled surface may extend from the tissue penetrating tip angled away from the center of the needle 250 to increase control and tissue penetration. A distal end of the needle flat surface 251 is partially exposed and shown extending distally from the tip of the guide element 212. As illustrated here, the needle flat surface 251 is lateral or generally perpendicular to the needle beveled surface 256 and may be described generally at the 3 o'clock position or perpendicular to the 90° radial.

The geometry of the guide element formed distal end 212 mediates a smooth transition from an exterior surface of the needle 250 to an exterior surface of the catheter 220. FIG. 10B provided an example of this where the needle flat surface 251 was in the 12 o'clock position and the distal end of the guide element 210 was mediating a transition between the needle beveled surface 256 to an exterior surface of the catheter lumen 221. FIGS. 17A and 17B illustrate a 3 o'clock orientation of the needle flat surface 251 and the guide element distal end 212 aligned accordingly. Here, the needle flat surface 251 does not extend to the tissue-penetrating tip 254 and generally terminates into the circumferential exterior surface of the needle proximal to the transition of the needle beveled surface 256. Also, the guide element distal end is in communication with the guide element cutout 226 at the catheter lumen distal end.

Considering FIGS. 17A and 17B, the a plane of the needle flat surface 251 will continue along a length of the needle. In an embodiment, the needle flat surface 251 may continue to the needle proximal end retained within the needle carrier 240. In other embodiments, the needle flat surface 251 may extend a length of the needle less than the entire length of the needle defined from the tissue-penetrating tip 254 to the needle proximal end. For example, the needle flat surface 251 may have a length from the needle carrier distal end 246 to a distal portion of the needle. The needle flat surface 251 may have a length defined by a proximal contact point of the guide element to the needle flat surface and extend to a distal portion of the needle sufficiently to mediate a smooth transitions of the intravascular access device distal end assembly.

Also referring to FIGS. 17A and 17B, the guide element formed distal tip 212 has a geometry confirmed to couple with the guide element cutout 226. For example, the guide element cutout may comprise a recess in the catheter tip where the guide element can be configured to seat or couple with when the guide element is in a ready-to-use configuration (e.g., prior to distal advancement).

FIGS. 17C and 17D are additional details of an embodiment regarding the alignment of the needle flat surface 251, the needle carrier 240, the second guide tube 235 (e.g., distal guide tube), the first guide tube 230 (e.g., proximal guide tube), and the arm 204 of the slide 202 positioned proximally as in the ready-to-use configuration. The handle 201 has been removed from view for illustration of the components therein. The slope of the needle carrier distal end 246 may be negative as the route of the guide wire is followed from the needle flat surface at the 6 o'clock position (or alternatively the 180° radial). The needle proximal end 252 is shown within the needle carrier and is superiorly situated above the second guide tube 235 within the needle carrier 240. The route of the guide element 210 tracks along the needle flat surface 251 then transitions through the catheter 220 and the needle carrier lumen 241 and into the distal guide tube 235 aligned along and near the lower portion of the handle.

The slide 202 shown near the proximal end of the intravascular device has an arm 204 that is coupled to the proximal end of the proximal guide tube 230. The length and orientation of the arm 204 may be dependent on the route of the guide element 210 and alignment of the proximal guide tube 230. For example, in FIG. 17C, the arm 204 extends further from the remainder of the slide to contact the proximal guide tube 230 when the needle flat surface 251 is in the 6 o'clock position. The guide tubes are below the needle 250 in the needle carrier 240 and therefore inferiorly disposed within the handle relative to the slot 207. The guide element tip 217 is shown between the tissue-penetrating tip 254 and the catheter tip 224. FIG. 17D is an expanded view of FIG. 17C where the relationship and details of the guide element route relating to the orientation of the needle flat surface 251, the needle carrier 240, the distal guide tube 235.

In some examples, the proximal guide tube 230 is configured to slidingly engage the distal guide tube 235 when the slide 202 is advanced. The guide element 210 may extend proximally through a lumen comprising the catheter assembly (e.g., from the catheter lumen), needle carrier 240, distal guide tube 235, and the proximal guide tube 230. The proximal end of the guide element elongate body may be within the proximal guide tube 230, inside of the handle. The proximal end or segment of the guide element may be coupled with the proximal guide tube 230 such that when the proximal guide tube 230 is advanced, retracted, or a combination thereof, the guide element is advanced (e.g., correspondingly with the proximal guide tube). For example, the slide 202 may engage the proximal guide tube 230 and advance distally within the handle causing the proximal guide tube 230 to advance into a lumen of the distal guide tube 235, and the guide element 210 may advance correspondingly with the proximal guide tube 230 such that the guide element distal tip is advanced from the guide element cutout of the catheter.

According to any of the embodiments disclosed herein, the orientation of the slot 207 of the handle 201 will remain through a top of the handle 201 such that the slot 207 can be considered superior to all components contained within or substantially within the handle. In some examples, the orientation and alignment of any of the elements described herein may be described relative to the superior location of the slot 207.

Referring to FIG. 17D, the distal guide tube 235 is positioned inferiorly within the handle and the guide element 210 extends from the distal guide tube 235 to the flat surface of the needle. In some examples, and as illustrated in FIG. 2C, the distal guide tube may comprise a length or segment that extends beyond the needle carrier 240 and/or distal end of the handle such that the guide element elongate body extends distally from the distal guide tube 235 along the plane of the flat surface of the needle.

FIG. 18 is a perspective view of the distal end of the catheter and access needle of FIG. 10A and FIG. 1A after the slide 50 has been moved from a proximal position as shown in FIG. 1A to a distal position where the guide element 120 is advanced distally along an outer wall 148 of the access needle to form a loop 128 in a position distal to the tip 144 of the access needle 140. In this exemplary embodiment, the loop 128 is formed approximately one inch beyond the distal end 144 of the needle 140. This view also shows how the upper and lower contours of the guide element 120 mate with the catheter guide element cut out 115 and the needle outer wall 148. As such, the guide element 120 may be advanced along an outer surface of an access needle to form a loop 128 beyond the distal end 144 of the needle 140. The deployed guide element 120 may form a partial, complete loop once or more than one complete loop beyond the distal end of the needle. In terms of degree of revolution about the distal end of the needle, a loop 128 may be more than 90 degrees, more than 180 degrees, more than 270 degrees or more than 360 degrees in the case of one full or more than one full revolution. By way of example, the loop 128 in FIGS. 18 and 19 is more than 270 degrees. Still further, exemplary diameters for a loop 128 range from 1 mm to 3 mm.

In some examples, the loop 128 may be a distal segment of the guide element configured to be flexible and provide the loop when the guide element 120 is advanced distally beyond the tissue penetrating tip 144. As described herein, when the guide element is to be retracted, the loop 128 (e.g., distal segment of the guide element) may be sufficiently flexible to be withdrawn into the catheter lumen and transition may to a sufficiently straight configuration without displacement or inadvertent impact to the position of the catheter within the vessel.

FIG. 19A is an enlarged view of the guide element loop of FIG. 18. The loop 128 is [0118] formed in the distal portion of the guide element 120 when deployed beyond distal most end 144 of access needle 140. This view also shows additional exemplary shapes for the guide element upper surface contour 124, lower surface contour 125, right side contour 127 and the left side contour 126. The upper and lower surface contours 124, 125 of guide element 120 conform to the space between catheter lumen inner wall and outer wall 148 of needle 140. In FIGS. 19B and 19C, additional details of shown of an exemplary guide element engagement with the guide element cutout of the catheter distal end. As described herein, the guide element can comprise a formed distal tip 217 with an elongate body 233 extending distally from the formed distal tip 217. In FIG. 19B, the formed distal tip 217 is attached, molded, or otherwise coupled with the elongate body 233 at 237. In some examples, the guide element formed distal tip 217 may be molded, manufactured or otherwise integrated with the guide element elongate body 233. FIG. 19B also shows a detail of the confirming geometry of the guide element distal tip 217. In this example, the guide element distal tip 217 comprises a proximal engagement feature 234 configured to seat or couple with the guide element cutout of the catheter tip.

In FIG. 19C, the guide element has been advanced distally from the guide element cutout 226 such that the guide element formed distal tip 217 is distal to the tissue penetrating tip 254 and has curled to form an atraumatic tip (e.g., when within the vessel). The guide element flat surface 211 is shown at the interior of the curl of loop formed by the guide element distal segment.

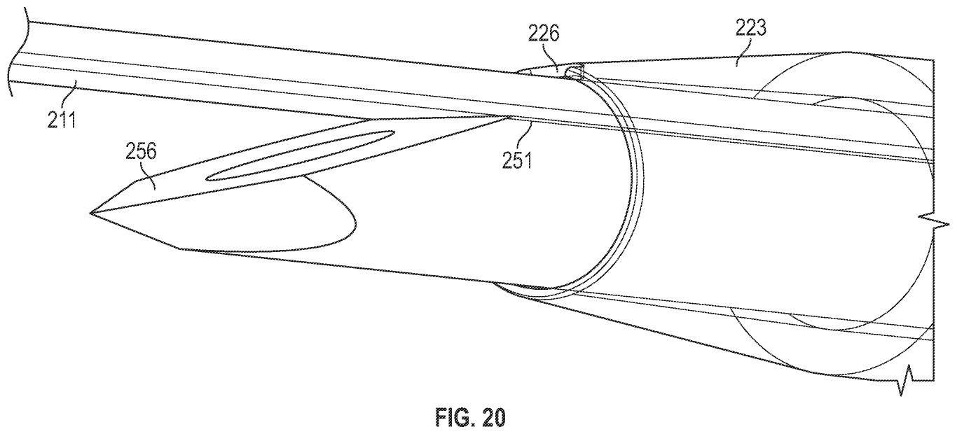

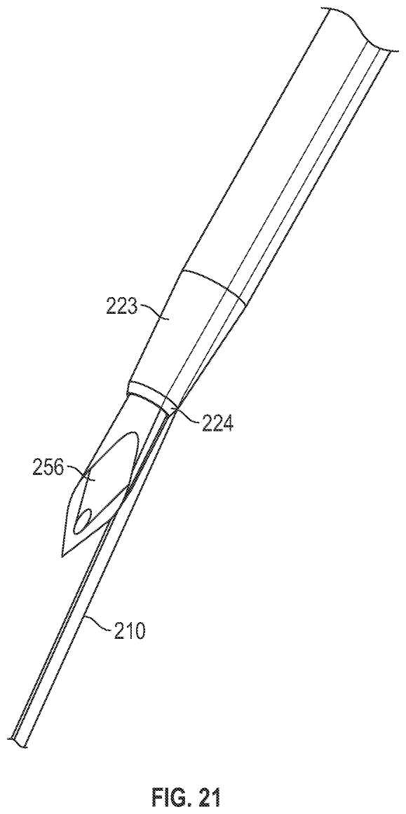

FIG. 20 is an enlarged view of the guide element distal end 212 deployed beyond the tissue-penetrating tip 254 after the slide 202 have been advanced towards a distal end of the handle 201 as shown in FIG. 1B. The guide element flat surface 211 is visible above the needle beveled surface 256 after sliding longitudinally across the needle flat surface 251. The cutout 226 at the catheter tip is no longer contacting the guide element distal end while the guide element is advanced. As seen here, the guide element 210 and needle flat surface 251 are oriented near a 12 o'clock position.

FIG. 21 is an enlarged perspective view of the needle distal end 246 with the guide element distal end 212 deployed beyond the tissue-penetrating tip 254. This embodiment illustrates a 3 o'clock orientation of the needle flat surface 251 and the guide element 210. In this orientation, the guide element distal end 212 extends beyond the tissue-penetrating tip 254 on a lateral side at 3 o'clock as illustrated in FIGS. 17A and 17B. While not illustrated, the guide element distal end curves after deployment beyond the tissue-penetrating tip 254 forming a loop.

In some embodiments, the curve of the guide element distal end 212 is formed as the guide element tip 217 curls in the direction of the tissue-penetrating tip 254. For example, as shown in FIG. 18, the guide element 120 is oriented at a 12 o'clock position and the tissue-penetrating tip 144 is below the guide element 120. Accordingly, the guide element tip 123 curls downward towards the tissue-penetrating tip 144. As another illustrative example, where the guide element 210 is oriented at the 3 o'clock position, the guide element tip curls laterally towards the tissue-penetrating element. In general, the guide element curls towards the needle.

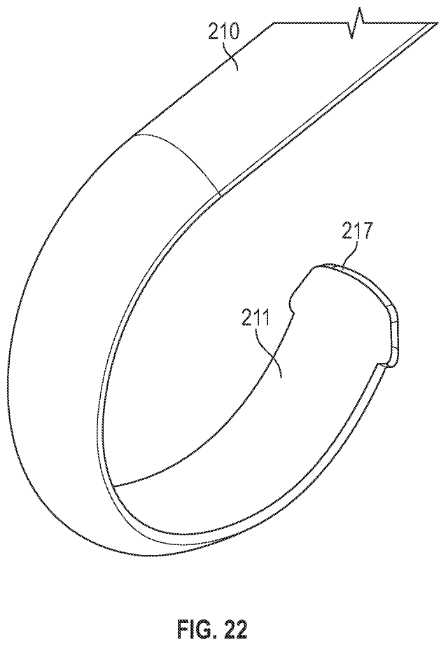

The deployed guide element 210 may form a partial, complete loop once or more than one complete loop beyond the distal end of the needle. FIG. 22 illustrates an embodiment wherein the guide element flat surface 211 is the interior side guide element loop. In terms of degree of revolution about the distal end of the needle, a loop may be more than 90 degrees, more than 180 degrees, more than 270 degrees or more than 360 degrees in the case of one full or more than one full revolution. An example of the guide element tip 217 geometry is shown optimized for smooth transition between the needle 250 and the catheter exterior surface.

In some embodiments, the guide element 120 may be formed from PTFE, nylon or another suitable polymer. Additionally or optionally, the guide element 120 may include an additive to enhance visibility under ultrasound guidance. Exemplary additives include, by way of example and not limitation, titanium oxide or barium. Still further, it to be appreciated that a wide array of materials may be used to form the guide element such as a number of polymer blends as well as metals, entirely or partially or of alloys of metals or shape memory materials including metallic and polymer based shape memory materials or of Nitinol or Elgiloy or the like. Still further the dimensions and contours illustrated in the various embodiments of the guide element may also vary along the length of the guide element. For example, the distal most portion of the guide element may have contours and dimensions that are directed to column strength. A mid portion of the guide element 120 may be designed to adapt for the transition from the housing to the catheter lumen without buckling. Still further transitions and variations of contour are possible not only to the guide element portion within the guide catheter but also the interior lumen wall of the catheter may be adapted to aid in the use of the various guide element embodiments. As such, in still further embodiments, the guide element distal tip design forms a uniform transition with the tubular catheter body distal end for maintaining ease of catheter insertion. Additionally, the guide element 120 may have a varying thickness proximal to the catheter hub to assist with column strength. In some embodiments, the distal face of the slide will mate with the proximal face of the needle carrier located inside the housing.

In some embodiments, the guide element 210 may be formed from PTFE, nylon or another suitable polymer. Additionally or optionally, the guide element 210 may include an additive to enhance visibility under ultrasound guidance. Exemplary additives include, by way of example and not limitation, titanium oxide or barium. Still further, it to be appreciated that a wide array of materials may be used to form the guide element such as a number of polymer blends as well as metals, entirely or partially or of alloys of metals or shape memory materials including metallic and polymer based shape memory materials or of Nitinol or Elgiloy or the like. Still further the dimensions and contours illustrated in the various embodiments of the guide element may also vary along the length of the guide element. For example, the distal most portion of the guide element may have contours and dimensions that are directed to column strength. A mid portion of the guide element 210 may be designed to adapt for the transition from the housing to the catheter lumen without buckling. Still further transitions and variations of contour are possible not only to the guide element portion within the guide catheter but also the interior lumen wall of the catheter may be adapted to aid in the use of the various guide element embodiments. As such, in still further embodiments, the guide element distal tip design forms a uniform transition with the tubular catheter body distal end for maintaining ease of catheter insertion. Additionally, the guide element 210 may have a varying thickness proximal to the catheter hub to assist with column strength. In some embodiments, the distal face of the slide will mate with the proximal face of the needle carrier located inside the housing.

In any embodiment described herein, the guide element may be composed of metallic materials, polymeric materials, or a combination thereof. For example, composition of guide element material may relate to characteristics and utility of the guide element function. The size and geometry of the guide element may also be determined based on optimal function of the guide element within the intravascular access device or the function of the guide element within the vasculature of a patient. Cross sectional geometry of the guide element may range from substantially circular or substantially elliptical to semicircular. For example, a guide element having a semicircular cross sectional geometry may be a semicircle. Another example of the guide element semicircular cross sectional geometry may be greater or larger than a semicircle, but less than a continuous and complete circle or ellipsis. In some embodiments, the shape and geometry of the guide element is based on an optimum moment of inertia of the guide element geometry. An exterior surface of the guide element may have an arc based on the internal surface of the catheter lumen. An surface of the guide element most proximal to the exterior surface of the needle may be considered a guide element interior surface. The guide element interior surface may be flat or substantially flat corresponding to the area of the guide element that contacts the exterior needle surface.

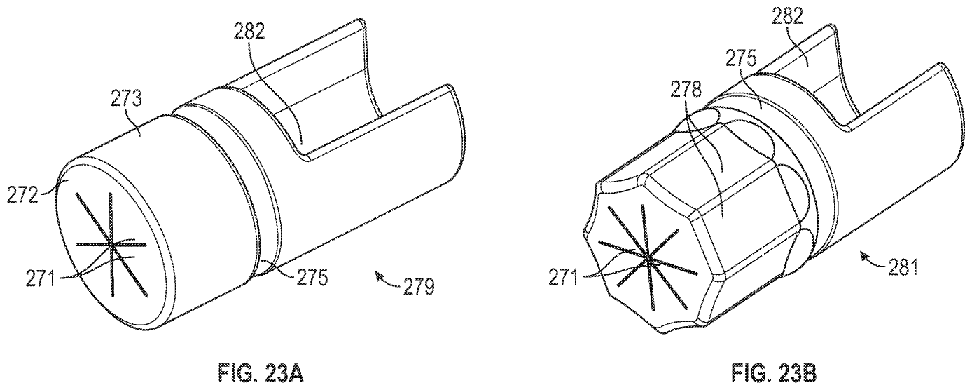

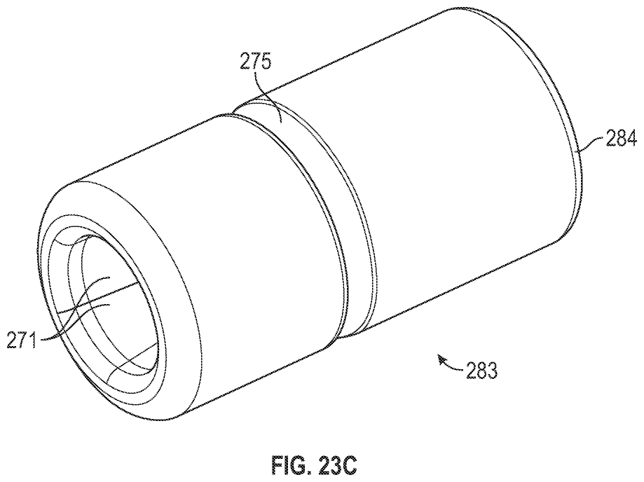

In some embodiments, a blood control valve is positioned inside the tubular catheter proximal hub. FIGS. 23A to 23C are an enlarged views of blood control valves inside the tubular catheter proximal hub according to some embodiments. FIG. 23A has a plurality of segments 271 on a distal surface 272. The plurality of segments can converge on a center of the distal face and are displaced as the catheter is advanced into vasculature of a patient. The circumferential valve body exterior 271 corresponds to a circumferential interior of the catheter proximal hub 222. A channel 274 around the circumference of the valve body 274 engages a corresponding element (e.g., a toroidal ridge) on the interior surface of the catheter proximal hub 222 to retain the valve in place until deployment. FIG. 23B is an enlarged perspective view of another homeostasis valve embodiment having a non-uniform exterior surface. In particular a distal portion of the valve body may have a series of longitudinal channels or depressions about the exterior surface. The channels 271 can be configured to increase a volume to the interior of the catheter proximal hub 222.

In some examples, the blood control valve may comprise one or more features to accommodate one or more elements of the intravascular access device. For example, and as illustrated by FIGS. 23A and 23B, the proximal end of the blood control valve body can comprise a recess configured to accommodate or receive the distal end of a needle carrier that may extend beyond the distal end of the handle. In some examples, the proximal send of the blood control valve may be a continuous as illustrated in FIG. 23C at 284 and shown in FIG. 2C.

In this configuration, the needle carrier may comprise a different distal configuration, shortened length, or combination thereof where the needle carrier is positioned some distance proximally from the blood control valve 283. For example, the needle carrier does not contact the blood control valve. FIG. 23C also illustrates an example of the distal end open with the segment 271 biased inward to the interior of the valve thereby opening the valve for a flow of blood to pass through an interior lumen of the valve. For example, in this configuration, the valve may be opened or actuated by a peripheral element such as a male segment of a luer lock coupled to the catheter hub and biasing the valve distally against a feature of the catheter hub causing the segments 271 to open.

In some examples, the blood control valve (e.g., 279, 281, 283) is positioned within ta catheter hub and configured to prevent, reduce, inhibit, or otherwise control a flow of blood from within the vessel through the catheter lumen and to an additional peripheral element (e.g., a male luer lock). Accordingly, the distal end of a blood control valve can be configured to contact or be contacted by a peripheral medical device or coupling to advance the blood control valve and open or initiate a flow of blood through the valve.

According to some embodiments, when the intravascular access device is used, the tissue-penetrating tip 254 enters the vasculature of a patient and fluid from within the vasculature may flow through the needle lumen into the catheter proximal hub to indicate the location of the tissue-penetrating tip 254 in the vasculature. A volume of fluid may increase within the catheter proximal hub and pressure therein may increase accordingly. Where the pressure of the fluid within the catheter proximal hub and needle lumen increased, deployment of the catheter may require additional force for advancement of the catheter lumen into the vasculature. The channels of the valve body increase the capacity of the catheter proximal hub and reduce the need for added pressure allowing for a smooth deployment of the catheter into the patient's vasculature and reducing potential for damage or injury associated with forced deployment of the catheter against the fluid pressure therein.

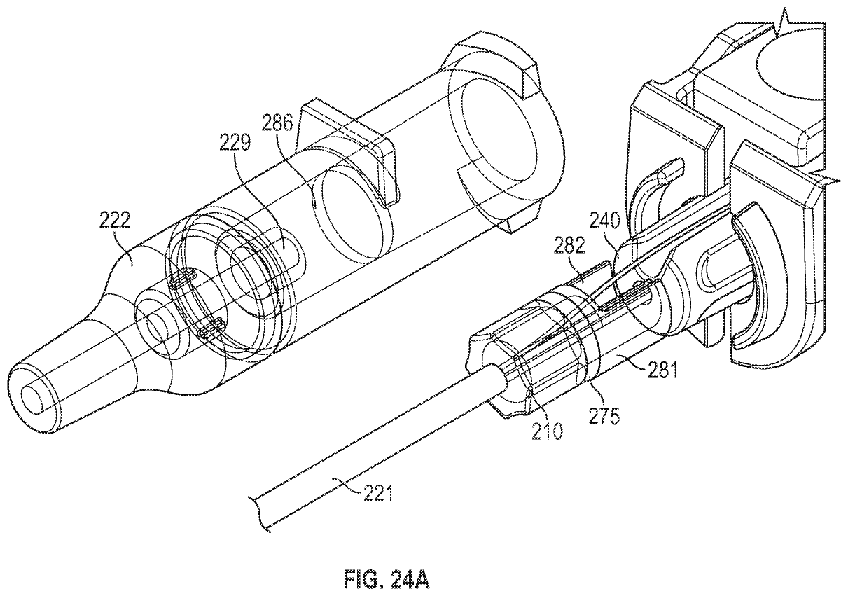

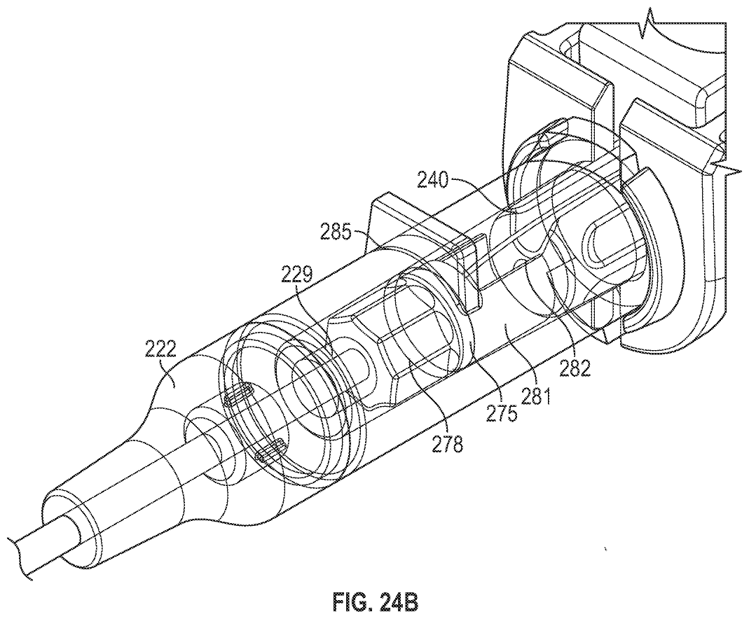

FIGS. 24A and 24B illustrate examples of the catheter hub configuration at a distal end of the intravascular access device. Referring to FIG. 24A, the catheter hub 222 is shown separate from the intravascular access device. Details of the catheter hub 222 are shown to include a valve control feature 229 configured to contact the segments 271 of a blood control valve (e.g., 279, 281, 283) when the blood control valve is advanced distally within the catheter hub 222. A blood control valve engagement feature 286 is visible and can be configured to engage a blood control valve, for example, at 275 to retain the blood control valve is an open position when the blood control valve has been sufficiently advanced within the catheter hub 222. In some examples, the blood control valve engagement feature 286 may be configured to retain the blood control valve in a closed position and distal advancement of the blood control valve may require distal displacement of the blood control valve from a coupling between the blood control valve engagement feature 286 and the corresponding engagement feature 275 of the blood control valve.

As will be appreciated by the various views of the figures, aspects of the present invention provide for improved methods, systems, and assemblies for performing venipuncture, in particular for placing intravascular catheters at a target location in a patient's vein. While the methods, systems, and assemblies will be particularly useful for placement of peripheral venous catheters, such as by placement in a vein on a hand or an arm, they can also be useful with placement of a central venous catheter by insertion into a central vein, such as the internal jugular vein on the neck or the subclavian vein on the chest. Still further, aspects of the vascular access methods, systems, and assemblies may be useful for placement of a catheter in a central or other artery.

In FIG. 24B, the catheter hub 222 is positioned or coupled with the distal end of the handle and shown as translucent to reveal an example configuration of the elements therein. For example, the blood control valve 281 is positioned within the catheter ub 222 and proximally adjacent to the valve control feature 229. The guide element can be seen transitioning through the handle and through the blood control valve 281.

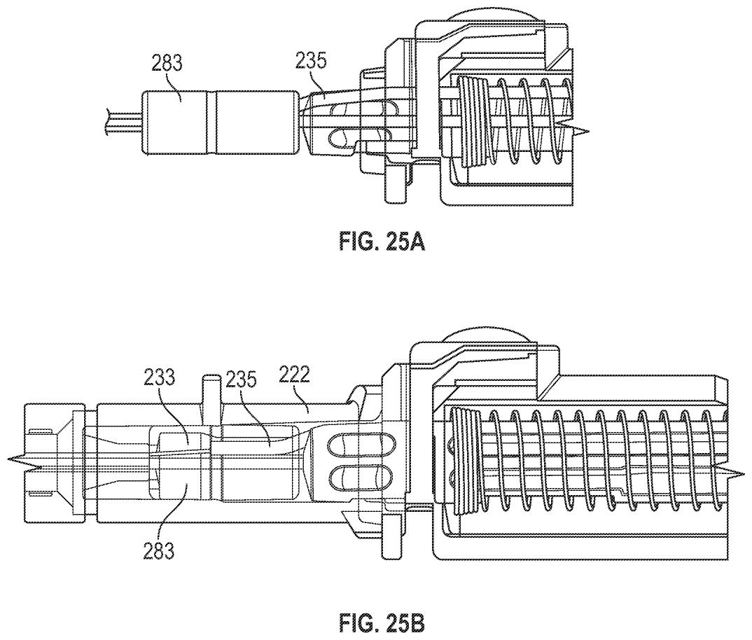

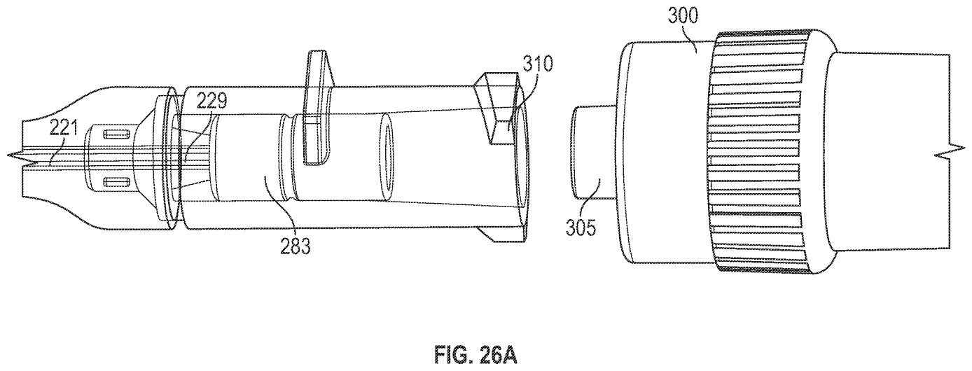

Additional detail and examples of the configuration within the catheter hub 222 at the distal end of the handle are shown in FIGS. 25A and 25B. Here, as previously illustrated in FIG. 2C, the distal guide tube 235 is shown routing the guide element elongate body 233 from the handle to the blood control valve 283 where the guide element elongate body 233 continues within the catheter lumen along the needle flat surface.

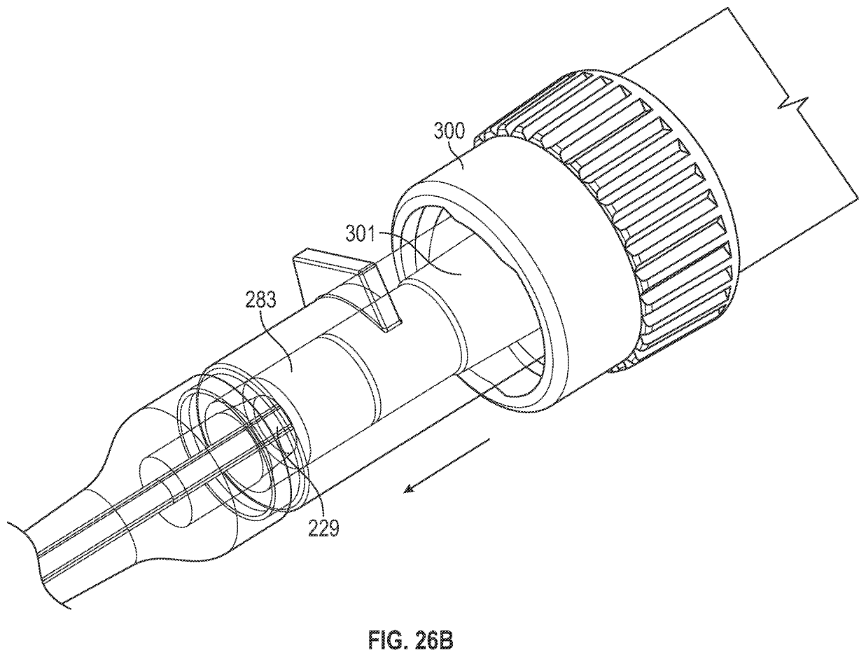

FIGS. 26A and 26B show an example engagement or coupling of a peripheral medical device with the catheter hub 222. For example, a male luer lock 300 having an engagement feature 305 can be configured to engage or contact the blood control valve 283 and advance the valve distally to engagement feature 229 such that the valve opens to allow a flow of blood therethrough. Referring to FIG. 26A, the male luer lock 300 has not yet contacted the blood control valve 283 or catheter hub 222, but is positioned or aligned with the proximal opening 310 of the catheter hub 222. Distally, the catheter lumen 221 is shown through the translucent catheter hub 222 to highlight an example of a path for the blood flow from the vessel. In FIG. 26B, the valve has been opened or transitioned from a closed state as illustrated by FIG. 26A, where the engagement feature 305 of the male luer lock 300 has engaged the proximal end of the blood control valve 283 and advanced the valve distally against the engagement feature 229 that is shown to open the distal surface of the valve completing a path or lumen for blood to flow from the catheter lumen 221 through the valve and to a coupled peripheral medical device.

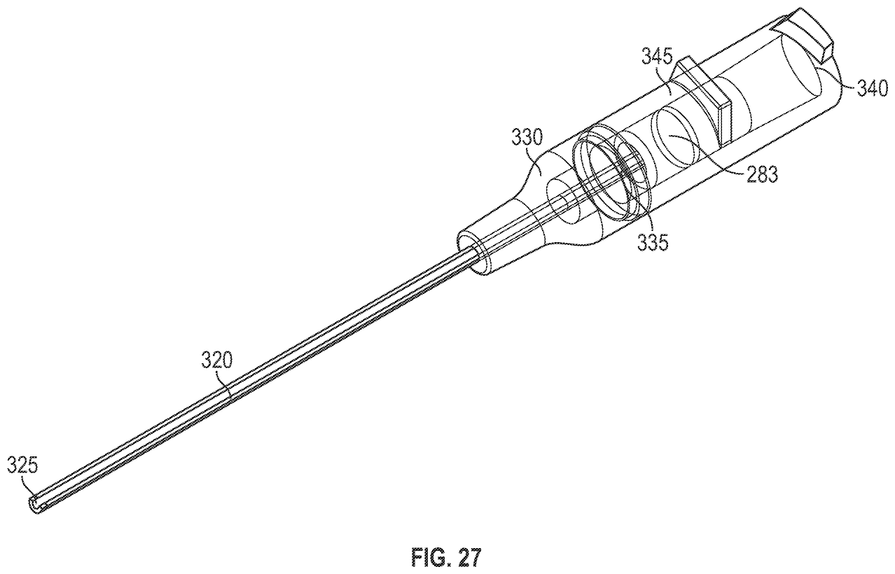

In some examples, a catheter assembly as illustrated in FIG. 27 can comprise a catheter lumen 320 having an interior and exterior wall. The distal tip of the catheter comprises a guide element cutout 325 configured to receive or couple with a guide element (e.g., guide element formed tip). The catheter lumen 320 may extend proximally from the distal tip with the guide element cutout 325 to a catheter hub 330 wherein a blood control valve 283 can be positioned and configured to selectively control (e.g., prevent or allow) a flow of blood from the vessel through the catheter lumen 320 and out the distal opening 340 when the catheter hub is coupled with a peripheral medical device. The blood control valve 283 can be configured to advance within the catheter hub when biased distally. For example, a male luer lock can couple with the distal end of the catheter hub 320 and be configured to advance the blood control valve distally against an engagement feature 335 of the catheter hub 330. The engagement feature may be adjacent to the distal surface of the blood control valve and can be configured to open the distal surface of the valve when the valve is based distally within the hub.

In use, an intravascular access device can be configured to access and position (e.g., insert) a catheter into a vessel of a patient. A catheter assembly having a catheter lumen extending between a distal tip with a guide element cutout and a catheter hub can be coupled to a distal end of a handle. A guide element with a formed distal tip can be configured to seat or couple with the guide element cutout and an elongate body of the guide element can extend along an exterior surface of an access needle disposed through the catheter lumen. In some examples, the entire guide element adjacent to the access needle is positioned exterior to the needle inside the catheter lumen. The proximal end of the needle can be coupled with a needle carrier coupled to an engagement element configured to proximally retract the needle, needle carrier and guide element proximally into the handle when an actuation button is depressed. A proximal and distal guide tube comprise a lumen through which the guide element elongate body extends proximally into the handle and a slider can be coupled with the proximal guide tube and be configured to advance the guide element distally. When the tissue penetrating tip of the needle is inserted into the vessel, flashback may be observed. For example, flashback may be observed in the catheter lumen, catheter assembly, etc. to indicate proper position within the vessel. The slider may be advanced distally thereby advancing the proximal guide tube and the guide element distal tip from within the guide element cutout. The guide element distal tip can be advanced beyond the tissue penetrating tip where the distal segment of the guide element may form an atraumatic tip (e.g., loop). Then, the catheter may be advanced along the guide element into the vessel. When the catheter is positioned within the vessel, the actuation button may be depressed and the actuation element can bias (e.g., force) the needle carrier proximally within the handle thereby retracting the needle and guide element. In some examples, a distal guide tube is correspondingly biased proximally whereby the distal guide tube slides proximally over the proximal guide tube.

In some examples, when the needle and guide element are to be retracted, actuation of the actuation element can be configured to withdraw the needle and the guide element simultaneously. In some examples, the needle and guide element may be selectively withdrawn such that the needle may be withdrawn when the actuation element is engaged, and the needle carrier is forced to the proximal end of the handle. Then the guide element may be withdrawn selectively after. In some examples, when the actuation element forces the needle carrier proximally within the handle, the needle may initially withdraw followed by the guide element that may be automatically withdrawn by the needle carrier or guide tubes being withdrawn. For example, when the actuation button is depressed and the actuation element biases the needle carrier proximally, the needle may withdraw with the needle carrier and the needle carrier may cause the distal guide tube, proximal guide tube, or a combination thereof to withdraw proximally within the handle causing the guide element distal tip to withdraw proximally. In some examples the guide element may straighten when it is withdrawn. For example, the distal segment of the guide element may be sufficiently flexible to transition from the loop (e.g., when distally advanced into the vessel) to a straight configuration at the distal end of the catheter lumen as to not inadvertently displace the catheter once it has been placed or positioned in the vessel.

The catheter hub may be uncoupled from the handle and the flow of blood can be restricted by the blood control valve within the catheter hub until a peripheral medical device or tube is coupled to the catheter hub and advanced the blood control valve distally within the catheter hub to activate or open the valve and initiate a flow of blood through the catheter assembly.

More generally, an intravascular catheter assembly in accordance with the principles of the present invention comprises a tubular catheter body, an access needle, a guide element, and a slide for deploying the guide element. The tubular catheter body has a distal end, a proximal end, at least one lumen therebetween and a guide element cutout formed in the distal end. The access needle has a tissue-penetrating distal tip and usually a lumen therethrough. In a first embodiment, the guide element is disposed in the spacing or the lumen between the access needle outer wall and the inner wall of the catheter lumen. In a second embodiment, the guide element is disposed outside and in parallel to the access needle riding along an axial groove, recess or guide structure or structures or guide elements formed in the catheter lumen inner wall. In all embodiments, the guide element has a distal tip configured to (a) conform to the shape and contour of the catheter distal tip in such a manner to form an smooth transition (see FIG. 10A) whereby the functionality of the needle catheter combination of providing access is maintained and (b) when extended distally of the catheter along the needle and when distal to the needle will form a partial or complete loop to facilitate advancement of the catheter in the vasculature.

In one embodiment, the slide of the intravascular vascular access assembly is slidably disposed over a support rail containing the guide element. The proximal end of the guide element is attached to the slide. In this way, the guide element may be distally advanced by sliding the slide forwardly or distally along the slot provided in the housing (FIG. 1). This movement moves the distal end of the guide element over the access needle outer surface in order to position the distal tip of the guide element beyond the distal end of the catheter. (FIG. 18). Once in this configuration, the access needle can be fully or partly retracted, or left in place without retraction, and the catheter and the distally projecting guide element can be advanced in tandem to position the distal end of the catheter body at a target site in the venous or other vasculature. By advancing the catheter and the projecting guide element in tandem, the guide element acts as a “fixed” guide element tip, further simplifying the catheter placement protocol. Moreover, the projecting loop of the distal portion of the guide element will aid in preventing or substantially inhibiting the distal tip of the catheter from sticking or kinking on an internal wall of the blood vessel.

In an additional aspect, the intravascular access device includes a housing having at its distal end a needle carrier attached to a proximal end of an access needle as shown in the various views of FIGS. 2A, 3, 4, 8 and 9. The access needle 140 is fixedly secured to the needle carrier 130 and the proximal end of the catheter is detachably secured to the distal end of the housing. In this way, the slide 150 can be used to advance the guide element beyond the distal ends of the catheter and the access needle, and the housing, access needle and guide element may be detached and removed from the catheter after the catheter is in place at a desired location in the vasculature.

Coupling the guide element 120 to the slide 150 can be accomplished in any manner which allows advancement or retraction of the slide to impart an equivalent advancement or retraction of the guide element through the space between the catheter lumen and the access needle outer wall. Advantageously, since the guide element is positioned outside of and along the exterior or outer wall of the access needle, the slide can be directly connected to a proximal end of the guide element with minimum interference from the access needle.

In some embodiments, the slide is connected to a proximal end of the first guide tube having the proximal end of the guide element retained therein. The slide may advance the first guide tube into the second guide tube causing simultaneous advancement of the guide element through the intravascular access device.

Another aspect of the present invention, the needle as described herein is selectively manufactured with the needle flat surface at a specific orientation on the exterior surface of the needle. Consideration of selection of an intravascular access device described here may regard attributes of the patient and the condition of their target vasculature. For example, depending on the intravascular access point, a vessel may have a non-linear path whereby it would be advantageous to select an intravascular access device with a needle flat surface and guide element orientation other than a 12 o'clock position to optimize opportunity of safe and successful deployment of the guide element and catheter into the patient's vasculature. Another example may include limiting patient anatomy or anatomical structures promoting selection of a needle flat surface and guide element orientation that would allow the least obstructed path for deployment of the guide element into the vasculature. Considering a common orientation of a needles having a tissue penetrating element, insertion generally proceeds with the tissue penetrating tip closest to the patient's exterior. Certain embodiments of the invention described herein provide optimal tissue penetrating tip orientation with selectable guide element positioning not limited to a specific position about the exterior surface of the needle.

In still another aspect of the present invention, a method for introducing an intravascular catheter to a target location in a blood vessel of a patient comprises penetrating a distal tip of an access needle which carries the catheter into the vein. A guide element is within a guide element cutout of the distal tip of the catheter. As a result introduction of the distal most end of the catheter into the blood vessel results in the distal end of the guide element also being positioned into the blood vessel. As such, once bleed back is detected and the needle/catheter combination is advanced such that the distal end of the catheter is within the vessel, the needle may be held stationary. With the needle position held stationary, the slide is advanced from the proximal position shown in FIGS. 1A and 2A into a distal position where the slider is in the distal portion of the slot along the top of the housing. The proximal to distal movement of the slider advances the guide element from the position within the guide cut out in the catheter distal tip (FIG. 10A) to the deployed condition with a curved portion, full or partial loop distal to the penetrating tip of the needle as best seen in FIGS. 18 and 19A. It is to be appreciated that the guiding element may be pre-shaped to form the loop 128 at a desired or selected distance from the needle distal end 144. In one embodiment, the loop 128 is formed at a minimum distance of about 1 inch or 25.4 mm. Other specific spacing between the needle distal tip and the loop may be provided based on application, clinical need and anatomical considerations of the target lumen.

After the guide element has been advanced, the access needle may optionally be retracted proximally leaving the guide element in place to aid in positioning of the catheter. Optionally, once the catheter is properly located within the blood vessel, the access needle and the guide element are withdrawn completely from the catheter, leaving the catheter in place for a desired medical protocol. In one embodiment, a spring-loaded retraction system or activation element 108 is activated by depressing the actuation button 106. Once the actuation button 106 is depressed or otherwise moved to cause the release of the needle carrier 130, the actuation spring 108 automatically drives the needle carrier 130, needle 140, slide 150 and guide element 120 proximally to withdraw the access needle 140 and guide element 120 from the patient and at least partially or completely into the housing 101. Thereafter the catheter hub 110 is detached from the distal end 103 of the handle or housing 101 leaving the catheter hub 110 in place for a desired medical protocol as mentioned above. The used housing 101 may then be disposed of using appropriate means.

A spool containing a surplus length of guide element may provide for augmentation of a length of the guide element. The spool may be coupled to the handle or separately in communication with the handle allowing a sufficient portion of excess guide element available for advancement into the patient's vasculature after the tissue-penetrating tip has successfully been inserted. For example, the tissue-penetrating tip may be inserted at an anatomical location distant from a desired target deployment location of the catheter. In such an example, the guide element may require a length sufficient to travel through extended sections of the patient's vasculature to the desired catheter deployment location. The guide element may be repeatedly advanced by the slide where the slide is depressed near a proximal end of the handle to engage the guide element. The slide is then advanced towards a distal end of the handle continuously depressed until it reaches the distal most portion of the slot. The slide may be released and slid back to the proximal end of the slot to be depressed and advanced distally. This process may be repeated until a sufficient amount of guide wire is deployed. In some embodiments, the handle may be removed proximally while the guide wire remains partially inserted into the patient's vasculature and an additional catheter or length of catheter may be slide over the guide element further into the vasculature to a desired catheter deployment location.

A needle carrier retention mechanism may be positioned within the body of the handle and support retention of the needle carrier after it has been retracted towards the proximal end of the handle. The needle carrier retention mechanism may engage a proximal end of the needle carrier or corresponding element disposed thereon to retain the needle carrier towards the proximal end of the handle and preventing oscillation of the actuation spring and needle carrier assembly.

Additional aspects of the construction and operation of a catheter placement device 100 which includes a housing or handle 101 having mechanism for advancing a guide structure or guide element which carries the catheter where the handle is adapted to automatically retract both the access needle and the guide structure or guide element from the catheter after the placement procedure is complete, a button activated automatic needle and guide withdrawal assembly are described in U.S. patent Publication US 2008/0300574 and U.S. Pat. No. 9,522,254, each of which is incorporated herein by reference in their entirety.

Still other details of representative intravascular catheter insertion devices and methods are described in U.S. Pat. Nos. 5,704,914 and 5,800,395 and in U.S. patent Publications US 2010/0094310; US 2010/0210934; and US 2012/0197200, the full disclosure of each of these are incorporated herein by reference in their entirety.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

When a feature or element is herein referred to as being “on” another feature or [0153] element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-43. (canceled)

44. An intravascular access device, comprising:
a handle having a proximal end and a distal end, a slot extending from the proximal end to the distal end;
a catheter having a proximal catheter hub and a distal catheter lumen, the proximal catheter hub releasably engaged to the distal end of the handle;
an access needle extending proximally from within a needle carrier through the catheter lumen, the access needle having at least one flat surface extending longitudinally along a perimeter of the access needle, and a tissue-penetrating tip extending distally beyond the catheter lumen;
at least one guide tube within the handle, the guide tube having a lumen therethrough and a distal end in communication with a lumen extending through a distal segment of the needle carrier;
a guide element having at least one longitudinal flat surface, the guide element extending from the proximal end of the handle through the at least one guide tube and needle carrier, a distal portion of the guide tube disposed within the catheter lumen, wherein the longitudinal flat surface of the guide element is in contact with the at least one flat surface of the access needle; and
a slide extending through the slot, the slide in communication with a proximal end of the guide element so that distal advancement of the slide advances a distal tip portion of the guide element from a position adjacent to a distal end of the catheter lumen along the at least one flat surface of the access needle.

45. The intravascular access device of claim 44, wherein the at least one flat surface of the access needle extends longitudinally down an exterior surface of the access needle.

46. The intravascular access device of claim 45, wherein the guide element is disposed through a path defined by the at least one flat surface of the access needle, a distal guide tube coupled to the needle, and a proximal guide tube axially aligned with the at least one flat surface of the access needle.

47. The intravascular access device of claim 45, wherein the at least one guide tube is aligned parallel to a plane of the at least one flat surface of the access needle.

48. The intravascular access device of claim 44, wherein when the slide is in a distal most position a distal portion of the guide element curves beyond the tissue-penetrating distal tip of the access needle.

49. The intravascular access device of claim 44, wherein when the slide is in a distal most position a distal portion of the guide element curves from a position defined by a plane of the at least one flat surface of the access needle, beyond the tissue-penetrating distal tip of the access needle.

50. The intravascular access device of claim 44, further comprising an access needle lumen, wherein a plane of the at least one flat surface of the access needle is perpendicular to a radius of the access needle lumen.

51. The intravascular access device of claim 44, further comprising a blood control valve is disposed within the proximal catheter hub, the blood control valve having a body with a distal end and a proximal end and a lumen within the body extending from the proximal end to the distal end.

52. The intravascular access device of claim 51, the distal end of the blood control valve comprising a surface extending across the lumen, the surface comprising a plurality of slots.

53. The intravascular access device of claim 51, the proximal end of the blood control valve body comprising a perimeter surface on a proximal end configured to engaged with a portion of a Luer to advance the blood control valve distally within the catheter hub displace the plurality of slots.

54. The intravascular access device of claim 53, wherein the proximal end perimeter is continuous on a single plane.

55. The intravascular access device of claim 53, wherein the proximal end perimeter comprises a recess into an exterior of the valve body.

56. The intravascular access device of claim 55, wherein in the recess is configured to accommodate a distal end of a distal guide tube, a segment of the guide element body, or distal end of the needle carrier.

57. The intravascular access device of claim 55, wherein in use when the needle carrier is in a distal most position the needle carrier is proximal to the blood control valve proximal end without contacting the blood control valve.

58. The intravascular access devices of claim 44, wherein the tissue-penetrating tip defines a distal terminus of a beveled surface of the access needle.

59. The intravascular access device of claim 44, wherein the at least one flat surface is offset on the perimeter of the access needle at a degree relative to the position of the tissue-penetrating tip.

60. The intravascular access device of claim 44, further comprising an actuation button coupled to the needle carrier, and an actuation element exerting a force on the needle carrier towards the proximal end of the handle, wherein when the actuation button is depressed the actuation element displaces the needle carrier and access needle toward the proximal end of the handle.

61. The intravascular access device of claim 44, wherein a plane of the at least one flat surface of the access needle is perpendicular to any radial extending outward from a center of the access needle.

62. The intravascular access device of claim 44, wherein the catheter is configured for use as a catheter component of an infusion set, a catheter component in a blood collection set, a catheter component in a safety winged blood collection set, or a catheter component in an intravenous catheter.

63. The intravascular access device of claim 44, further comprising a spool of guide element in communication with the proximal end of the intravascular device, wherein a length of the guide element is contained within the spool.

64. The intravascular access device of claim 44, wherein the guide element is made entirely or partially of a metallic material, polymeric material, or a combination thereof.

65.-111. (canceled)
